(19) United States
(12) Patent Application Publication
(10) Pub. No.: US 2023/0375543 A1
(43) Pub. Date: Nov. 23, 2023

(54) ASSAY SYSTEM FOR MULTIPLE ANALYTES

(71) Applicant: UniQ Biotech Ltd., Delta (CA)

(72) Inventors: Yunchuan ZHANG, Coquitlam (CA); Danyang SHEN, Victoria (CA); Zelin FU, Burnaby (CA)

(21) Appl. No.: 18/319,302

(22) Filed: May 17, 2023

Related U.S. Application Data
(60) Provisional application No. 63/342,962, filed on May 17, 2022.

(51) Int. Cl. G01N 33/543; G01N 33/569

(52) U.S. Cl. CPC G01N 33/54389; G01N 33/56983; G01N 2333/165

(57) ABSTRACT

An assay system for detecting the presence or absence of at least a first analyte and a second analyte in a sample is disclosed. The assay system comprises an assay device and a separate label solution comprising a detection molecule. The assay device comprises: a first detection region for receiving the sample in a vertical direction perpendicular to a longitudinal axis; a second detection region for receiving the sample from the first detection region in a horizontal direction parallel to the longitudinal axis; a first immobilized molecule in one of the first and second detection regions configured to bind to either the detection molecule or the first analyte; and a second immobilized molecule in the other one of the first and second detection regions and configured to bind to the second analyte to generate a complex, wherein the detection molecule is configured to bind to the complex.

### RELATED PATENTS AND/OR APPLICATIONS

This application claims the benefit under 35 U.S.C § 119(e) of U.S. provisional application No. 63/342,962, titled “ASSAY SYSTEM FOR MULTIPLE ANALYTES”, filed on May 17, 2022, and incorporated herein by reference.

### BACKGROUND

Assay systems can be used to detect presence or absence of analytes of interest in a sample. Flow assays typically include an immobilized capture molecule bound to a substrate, wherein the immobilized capture molecule is designed to bind to the analytes of interest in the sample.

### SUMMARY

Assay systems may be able to detect the presence or absence of an analyte in a sample. Assay systems may also utilize a combination of vertical and lateral flow to detect one or more analyte of interest in a sample. However, such assays can be difficult to use, manufacture and store as they involve multiple detection molecules and the results can be challenging to interpret. Additionally, such assays can also provide inaccurate results and lead to false negatives or false positives that can mislead a user as to their current health status.

Other aspects and features of the present disclosure will become apparent to those ordinarily skilled in the art upon review of the following description of specific embodiments of the disclosure in conjunction with the accompanying figures.

In one embodiment, there is provided an assay system for detecting the presence or absence of at least a first analyte and a second analyte in a sample. The assay system comprises a label solution comprising a detection molecule and an assay device. The assay device comprises: a first detection region configured to receive the sample in a vertical direction perpendicular to a longitudinal axis of the assay device; a second detection region in liquid communication with the first detection region and configured to receive the sample from the first detection region in a horizontal direction parallel to the longitudinal axis; a first immobilized molecule immobilized in one of the first and second detection regions and configured to bind to either the detection molecule or the first analyte to indicate the presence or absence of the first analyte in the sample; and a second immobilized molecule immobilized in the other one of the first and second detection regions and configured to bind to the second analyte to generate a complex, wherein the detection molecule is configured to bind to the complex to indicate the presence or absence of the second analyte in the sample.

In another embodiment, there is provided a method of detecting the presence or absence of at least a first analyte and a second analyte in a sample using an assay system. The method comprises applying the sample in a vertical direction perpendicular to a longitudinal axis of an assay device to a first detection region of an assay device. The sample flows from the first detection region in a horizontal direction parallel to the longitudinal axis to a second detection region of the assay device. The method further comprises applying a label solution in the vertical direction to the first detection region. The label solution comprises a detection molecule. The label solution also flows from the first detection region in the horizontal direction to the second detection region. The assay device comprises: a first immobilized molecule immobilized in one of the first and second detection regions and configured to bind to either the detection molecule or the first analyte to indicate the presence or absence of the first analyte in the sample; and a second immobilized molecule immobilized in the other one of the first and second detection regions and configured to bind to the second analyte to generate a complex, wherein the detection molecule is configured to bind to the complex indicate the presence or absence of the second analyte in the sample.

In one embodiment, there is provided an assay system for detecting presence or absence of at least a first analyte and a second analyte in a sample. The assay system comprises a label solution comprising a detection molecule and an assay device. The assay device comprises: a first detection region configured to receive the sample in a vertical direction perpendicular to a longitudinal axis of the assay device; a second detection region in liquid communication with the first detection region and configured to receive the sample from the first detection region in a horizontal direction parallel to the longitudinal axis; a first immobilized molecule immobilized in one of the first and second detection regions and configured to bind to either the detection molecule or the first analyte to indicate the presence or the absence of the first analyte in the sample; and a second immobilized molecule immobilized in the other one of the first and second detection regions and configured to bind to the second analyte to generate a complex. The detection molecule is also configured to bind to the complex to indicate the presence or the absence of the second analyte in the sample.

The first immobilized molecule may be immobilized in the first detection region and the second immobilized molecule may be immobilized in the second detection region.

If the first immobilized molecule remains unbound by the first analyte after the sample is applied to the first detection region, the detection molecule may bind to the first immobilized molecule to generate a detectable signal indicating the absence of the first analyte in the sample. If the first immobilized molecule binds to the first analyte after the sample is applied to the first detection region, the detection molecule may not bind to the first immobilized molecule and may generate a null signal indicating the presence of the first analyte in the sample.

If the second immobilized molecule binds to the second analyte to generate the complex after the sample is applied to the second detection region, the detection molecule may bind to the complex to generate a detectable signal indicating the presence of the second analyte in the sample. If the second immobilized molecule remains unbound by the second analyte after the sample is applied to the second detection region, the detection molecule may not bind to any complex and may generate a null signal indicating the absence of the second analyte in the sample.

The first immobilized molecule may be immobilized in the second detection region and the second immobilized molecule may be immobilized in the first detection region.

The sample may be applied to the first detection region prior to the label solution being applied to the first detection region. Alternatively or additionally, the sample may be applied to the second detection region prior to the label solution being applied to the second detection region.

The first analyte may be an antibody.

The second analyte may be a viral particle or an antigenic portion thereof.

The first immobilized molecule may comprise: a protein, an antibody, an antigen-binding fragment of an antibody, an antigen, a peptide, a nucleic acid, or a combination thereof; or any molecule that can bind a protein, an antibody, an antigen-binding fragment of an antibody, an antigen, a peptide, or a nucleic acid.

The first immobilized molecule may comprise a peptide that binds to an anti-SARS-Cov-2 S-protein neutralizing antibody and an angiotensin converting enzyme 2 (ACE 2) protein. The second immobilized molecule may comprise a recombinant anti-SARS-Cov-2 antibody.

The detection molecule may comprise a binding moiety and a label moiety. The binding moiety may be a protein, an antibody, an antigen-binding fragment of an antibody, an antigen, or a peptide.

The label moiety may comprise a vat dye particle. The vat dye particle may comprise isatin, vat red 1, vat red 41, or vat orange 7.

The vat dye particle may be below a threshold size.

The vat dye particle may have a positively charged hydrophilic group and the binding moiety may be treated to have a negative charge. The vat dye particle may have a negatively charged hydrophilic group and the binding moiety may be treated to have a positive charge.

Each detection molecule may have more than one label moiety attached to one binding moiety.

In another embodiment, there is provided a method of detecting presence or absence of at least a first analyte and a second analyte in a sample using an assay system. The method comprises applying the sample in a vertical direction perpendicular to a longitudinal axis of an assay device to a first detection region of the assay device. The sample flows from the first detection region in a horizontal direction parallel to the longitudinal axis to a second detection region of the assay device. The method further comprises applying a label solution in the vertical direction to the first detection region. The label solution comprises a detection molecule and the label solution also flows from the first detection region in the horizontal direction to the second detection region. The assay device comprises: a first immobilized molecule immobilized in one of the first and second detection regions and configured to bind to either the detection molecule or the first analyte to indicate the presence or the absence of the first analyte in the sample; and a second immobilized molecule immobilized in the other one of the first and second detection regions and configured to bind to the second analyte to generate a complex. The detection molecule is configured to bind to the complex to indicate the presence or the absence of the second analyte in the sample.

The first immobilized molecule may be immobilized in the first detection region and the second immobilized molecule may be immobilized in the second detection region.

If the first immobilized molecule remains unbound by the first analyte after the sample is applied to the first detection region, the method may further comprise detecting a detectable signal generated by the detection molecule binding to the first immobilized molecule indicating the absence of the first analyte in the sample. If the first immobilized molecule binds to the first analyte after the sample is applied to the first detection region, the method may further comprise detecting a null signal generated by the detection molecule not binding to the first immobilized molecule indicating the presence of the first analyte in the sample.

If the second immobilized molecule binds to the second analyte to generate the complex after the sample is applied to the second detection region, the method may further comprise detecting a detectable signal generated by the detection molecule binding to the complex indicating the presence of the second analyte in the sample. If the second immobilized molecule remains unbound by the second analyte after the sample is applied to the second detection region, the method may further comprise detecting a null signal generated by the detection molecule not binding to any complex indicating the absence of the second analyte in the sample.

Applying the sample and the label solution to the first detection region may comprise applying the sample to the first detection region prior to applying the label solution to the first detection region.

The first analyte may be an anti-SARS-Cov-2 neutralizing antibody.

The second analyte may be a SARS-Cov-2 viral particle or an antigenic portion thereof.

The first immobilized molecule may comprise a recombinant receptor-binding domain (RBD) protein of a S-protein from a SARS-Cov-2 virus.

The second immobilized molecule may comprise a recombinant anti-SARS-Cov-2 antibody.

The second immobilized molecule may comprise a recombinant anti-SARS-Cov-2 N-protein antibody.

The binding moiety may be a recombinant ACE 2 protein.

The label moiety may comprise one or more of a dye particle, a vat dye particle, a colored particle, a colored bead, an enzyme, a substrate, a chromogen, a catalyst, a fluorescent compound, a chemiluminescent compound, a radioactive label, a colloidal metallic particle, a colloidal gold particle, a colloidal non-metallic particle, a stained microorganism, or a colored organic polymer latex particle.

The label solution may have a viscosity above a viscosity threshold.

The label solution may further comprise whey protein.

The first immobilized molecule may be immobilized in the second detection region and the second immobilized molecule may be immobilized in the first detection region.

### BRIEF DESCRIPTION OF THE DRAWINGS

In drawings which illustrate embodiments,

FIG. 1 is a schematic of an assay system according to an embodiment of the disclosure;

FIG. 2 is a cross-sectional side view of an assay device of the assay system of FIG. 1;

FIG. 3 is a top view of an assay device of the assay system of FIG. 1 including a housing;

FIG. 4 is a schematic of an assay system according to another embodiment;

FIG. 5 is a schematic of a method of detecting presence and absence of a first analyte and a second analyte using the assay system of FIG. 1;

FIGS. 6A-6D are schematics of an assay device of the assay system of FIG. 1 when used according to the method of FIG. 5; and

FIG. 7 is a schematic of a method of combining a label moiety and a binding moiety to form a detection molecule used in the assay system of FIG. 1.

### DETAILED DESCRIPTION

The present disclosure provides assay systems, assay devices, and methods for determining presence or absence of at least a first analyte and a second analyte in a sample utilizing a label solution separate from the assay device.

Terms defined herein are provided solely to aid in the understanding of the present disclosure and should not be construed to have a scope less than understood by a person of ordinary skill in the art.

Terms of degree such as “about”, “approximately” and “substantially” refer to the indicated value and to all values that are within experimental error or operational error of the indicated value (e.g. within the 95% confidence interval for the mean) or within 10 percent of the indicated value, whichever is greater. These terms may refer to a measurable value such as an amount, a temporal duration, etc. Unless otherwise required by context, singular terms such as “a” and “an”, are understood to include pluralities and plural terms are understood to include the singular. Any examples following the term “for example” or “e.g.” are not meant to be limiting or exhaustive. The terms “comprises”, “comprising”, “include”, “includes”, “including”, “contain”, “contains” and “containing” are meant to imply inclusion of the stated element or step but not to the exclusion of other elements or steps.

The term “analyte” refers to any substance or chemical constituent of a sample that is being detected. An analyte may be any substance for which there exists a mechanism for detecting the substance utilizing a specific binding interaction. For example, the analyte (or portion thereof) can be an antigen or hapten having at least one site for binding to a naturally occurring or synthetically derived antibody. As an alternative example, the analyte (or portion thereof) may be an antibody having at least one site for binding to a naturally occurring antigen or a synthetically derived receptor binding domain (RBD).

The term “antibody” refers to a protein that specifically binds to a particular epitope on at least one antigen. An antibody can be a polyclonal antibody, a monoclonal antibody, a naturally derived antibody, or a genetically engineered molecule capable of specifically binding the corresponding antigen. The term “neutralizes” or “neutralizing antibody” means an antibody that reduces a biological activity (eg. binding and/or infectivity) of the antigen to which the neutralizing antibody binds. The term “antigen” refers to any substance that specifically binds to an antibody.

The terms “binding”, “bind”, “bound”, “capable of binding”, or “configured to bind” may be used to refer to the physical or chemical interaction between two molecules, polypeptides, proteins, compounds or any combinations thereof to result in attachment thereof. The chemical interactions may be covalent bonds, such as ionic or non-ionic covalent bonds, or may be non-covalent bonds, such as bonds resulting from van der Waals forces, electrostatic forces, hydrophobic interactions, etc. The interactions can be either direct or indirect. Indirect interactions may be through, or due to the effect of, another molecule, polypeptide, protein, compound. Direct interactions may be interactions directly between two molecules, polypeptides, proteins, or compounds. The terms “specifically binds”, or “binds specifically” is a term understood in the art, and methods to determine the level of specific binding between two complementary molecules, polypeptides, proteins, or compounds are known in the art. Generally, when two complementary molecules, polypeptides, proteins, or compounds “specifically binds” to each other or “binds specifically” each other, the two complementary molecules, polypeptides, proteins or compounds binds to each other with greater affinity, avidity, more readily, and/or for a greater duration when compared to other substances.

The term “competes”, may be used to refer to a mechanism whereby a first molecule, polypeptide, protein or compound (or combinations thereof) binds to a second molecule, polypeptide, protein, compound (or combinations thereof) in a manner sufficiently similar in specificity as a third molecule, polypeptide, protein or compound (or combination binds to the second molecule, such that binding of the first molecule to the second molecule prevents binding of the third molecule to the second molecule and vice versa.

The term “polypeptide”, “peptide”, and “protein” may be used interchangeably to refer to chains of amino acids of any length and may comprise amino acids modified naturally or by intervention, such modifications including disulfide bond formation, glycosylation, lipidation, acetylation, phosphorylation, or any other manipulation or modification, such as conjugation or binding with a labeling component. Also included within the definition are polypeptides containing one or more analogs of an amino acid (such as unnatural amino acids, for example), as well as other modifications known in the art.

The expression “at least one of A or B” is interchangeable with the expression “A and/or B”. It refers to a list in which you may select A or B or both A and B. Similarly, “at least one of A, B, or C”, as used herein, is interchangeable with “A and/or B and/or C” or “A, B, and/or C”. It refers to a list in which you may select: A or B or C, or both A and B, or both A and C, or both B and C, or all of A, B and C. The same principle applies for longer lists having a same format.

Although the present invention has been described with reference to specific features and embodiments thereof, various modifications and combinations may be made thereto without departing from the invention. The description and drawings are, accordingly, to be regarded simply as an illustration of some embodiments of the invention as defined by the appended claims, and are contemplated to cover any and all modifications, variations, combinations or equivalents that fall within the scope of the present invention. Therefore, although the present invention and its advantages have been described in detail, various changes, substitutions, and alterations may be made herein without departing from the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

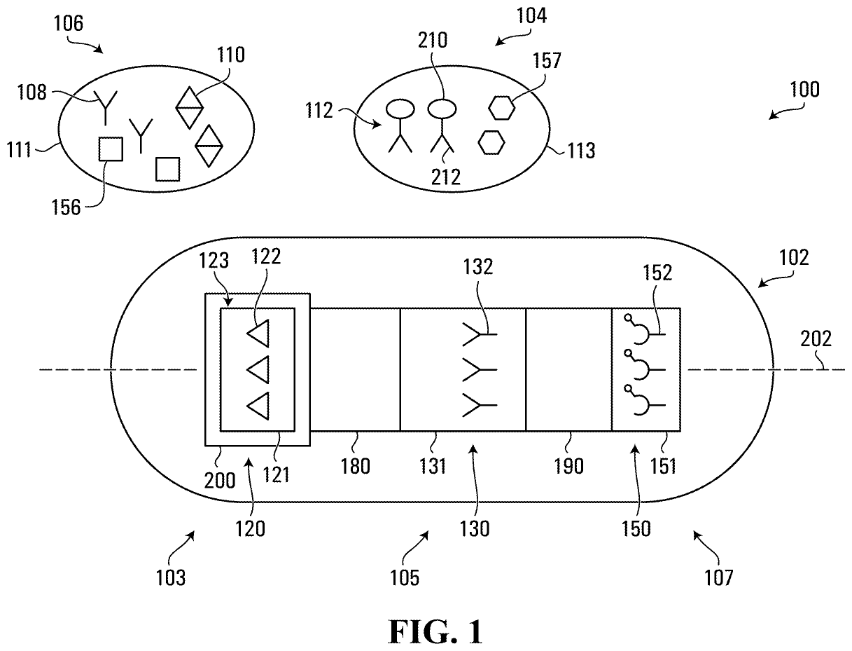

Referring to FIG. 1, an assay system according to an embodiment is shown generally at 100. The assay system 100 may be used to detect presence or absence of at least a first analyte 108 and a second analyte 110 in a sample 106. The sample 106 may be any biological sample such as cell samples, bacterial samples, virus samples, samples of other microorganisms, samples obtained from a mammalian subject, such as tissue samples, cell culture samples, stool or fecal samples, carcass swab samples, and biological liquid samples (e.g., nasal swab, nasopharyngeal swab, blood, plasma, serum, saliva, urine, cerebral or spinal liquid, and lymph liquid), environmental samples, air samples, water samples, dust samples and soil samples, and food samples.

The assay system 100 includes an assay device shown generally at 102 and a label solution shown generally at 104. The assay device has a longitudinal axis 202. In the embodiment shown, the assay device 102 includes a first portion 103, a second portion 105 and a control portion 107. In other embodiments, the assay device 102 may include fewer or additional portions, and may not have the control portion 107 or may have an additional third portion (not shown) for example.

The first portion 103 includes a first detection region 120 configured to receive the sample 106 and/or the label solution 104 in a vertical direction substantially perpendicular to the longitudinal axis 202 of the assay device 102.

The first detection region 120 includes a first material 121 to which a first immobilized molecule 122 is bound. The first material 121 may be any porous material suitable for use in flow-through or vertical flow assay devices that allows for at least (a) binding of the first immobilized molecule 122 thereto and (b) capillary action and transport of non-immobilized liquid (such as the sample 106 and the label solution 104 for example). In the embodiment shown in FIGS. 1-4, the first material 121 is a HiFlow™ nitrocellulose membrane manufactured by Millipore™, such as HF180 having a capillary flow rate of 180±45 sec/4 cm, HF135 having a capillary flow rate of 135±34 sec/4 cm or HF120 having a capillary flow rate of 120±30 sec/4 cm for example. Other nitrocellulose membranes that are may be used as the first material 121 include the Sartorius™ CN140 Membrane, ThermoFisher™ 88018, or Nupore™ FTCN-SH09. Generally, nitrocellulose membranes are manufactured with a range in measured properties (such as membrane thickness, weight, density, porosity, pore size, capillary flow rate for example) and may be selected depending upon the size of analytes to be detected, the size and type of molecules to be immobilized thereon, and the viscosity of the sample 106. In other embodiments, the first material 121 may include, for example, high density polyethylene, acrylic fiber, polyvinyl chloride, polyvinyl acetate, copolymers of vinyl acetate and vinyl chloride, polyamide, polycarbonate, polystyrene, untreated paper, cellulose blends, other cellulose derivatives such as cellulose acetate, fiberglass, cloth including natural and synthetic cloths, porous gels, porous fibrous matrixes, starch-based materials, and combinations or variations thereof. The material of the first material 121 may be selected such that the sample 106 and/or the label solution 104 (having a respective viscosity) travels an entire length of the first material 121 within a set amount time.

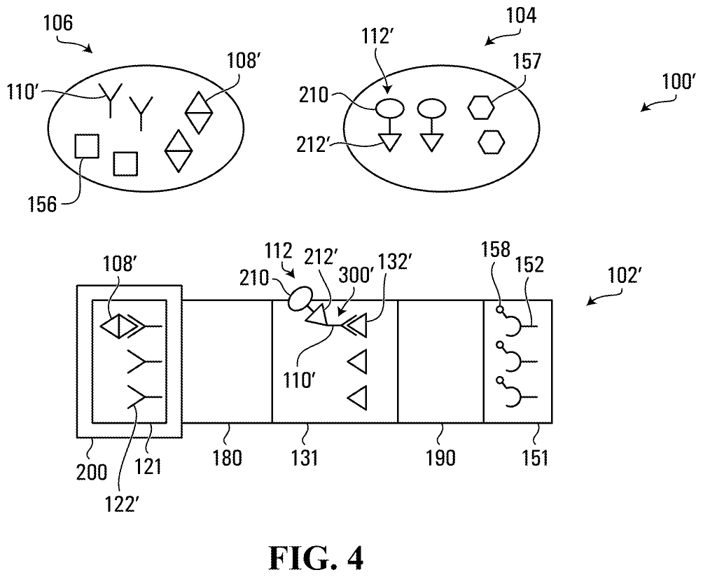

In the embodiment shown in FIGS. 1 and 4, the first immobilized molecule 122 is designed to be capable of specifically binding to the first analyte 108 in the sample 106 and of specifically binding to a detection molecule 112 in the label solution 104, but not both simultaneously, to indicate the presence or the absence of the first analyte 108 in the sample 106.

For example, in the embodiment shown in FIG. 1, when the first analyte 108 to be detected in the sample 106 is a naturally occurring neutralizing antibody capable of specifically binding to an antigen from the virus, bacteria, or other microorganisms, the first immobilized molecule 122 may be a recombinant or synthetically derived RBD protein which is an analog or a homolog of that antigen, while the detection molecule 112 includes a binding moiety 212 comprising a recombinant or synthetically derived antibody designed to also be capable of specifically binding to the immobilized recombinant RBD protein 122. As a more specific example, the assay system 100 may be designed to indicate the presence or absence of SARS-CoV-2 infection in a host and antibodies produced in response by the immune system of the host. In such embodiments, the first immobilized molecule 122 may be a recombinant RBD of the Spike protein (“S-protein”) from SARS-CoV-2 virus (SEQ ID NO: 2) to detect an anti-SARS-COV-2 S-protein antibody analyte 108 in the sample 106. Alternatively, as another specific example, the assay system 100 may be designed to indicate the presence or absence of an HIV infection in a host and antibodies produced in response by the immune system of the host. In such embodiments, the first immobilized molecule 122 may be a recombinant RBD of at least one envelope protein from HIV (such as a recombinant RBD of transmembrane glycoprotein gp36 from HIV-2, transmembrane glycoprotein gp41 from HIV-1 or transmembrane glycoprotein gp120 from HIV-0 for example) to detect an anti-HIV envelope protein neutralizing antibody analyte 108 in the sample 106. Alternatively, in the embodiment shown in FIG. 4, when the first analyte 108′ to be detected in the sample 106 is a naturally occurring antigen from a virus, a bacteria or another microorganism, the first immobilized molecule 122′ may instead be a recombinant or synthetically derived antibody designed to be capable of specifically binding to that antigen, while the detection molecule 112′ includes a binding moiety 212′ comprising a recombinant or synthetically derived RBD protein design to also be capable of specifically binding to the immobilized recombinant antibody 122′.

As the first immobilized molecule 122 is designed to be capable of specifically binding to either the detection molecule 112 or the first analyte 108, but not both simultaneously, the first analyte 108 and the detection molecule 112 compete with each other to bind to the first immobilized molecule 122 in the first detection region 120 when the first analyte 108 is present in the sample 106.

The first immobilized molecule 122 may be bound to the first material 121 using a variety of different ways known in the art, such as via covalent or non-covalent bonds (such as hydrophobic or electrostatic interaction, for example). For example, in embodiments where the first immobilized molecule 122 comprises a recombinant or synthetically derived RBD protein (shown in FIG. 1) or a recombinant or synthetic derived antibody (shown in FIG. 4), the first material 121 may include an ionic or anionic surfactant which partially denatures the amino acids, or the secondary, tertiary or quaternary folding structure(s) of the first immobilized molecule 122 to encourage the first immobilized molecule 122 to bind to the fibers of the first material 121 via hydrophobic interactions between the denatured amino acids and the fibers.

The first portion 103 also includes a deposit zone 123 where the sample 106 and the label solution 104 can be deposited onto the first material 121 in the vertical direction substantially perpendicular to the longitudinal axis 202 of the assay device 102. Once the sample 106 and/or the label solution 104 is deposited in the vertical direction in the deposit zone 123, the sample 106 and/or the label solution 104 flow from the first portion 103 in a horizontal direction substantially parallel to the longitudinal axis 202 to the second portion 105 of the assay device 102. The second portion 105 includes a second detection region 130 configured to receive the sample 106 and/or the label solution 104 from the first portion 103 in the horizontal direction substantially parallel to the longitudinal axis 202.

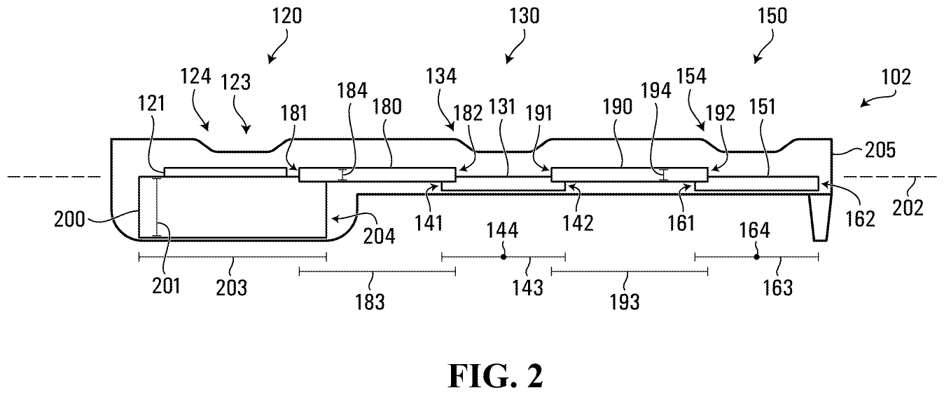

The second detection region 130 includes a second material 131 to which a second immobilized molecule 132 is bound. Referring to FIG. 2, the second material 131 has a first end 141, a second end 142, and a length 143 representing a distance between the first end 141 from the second end 142. The second immobilized molecule 132 may be attached to the second material 131 at a position 144 at a midway point along the length 143. For example, in the embodiment shown in FIG. 2, the length 143 is approximately 1.5 cm and the position 144 is approximately 0.75 cm from the first end 141. In other embodiments, the length 143 may range between approximately 0.25 cm and approximately 3 cm and the position 144 may correspondingly range between approximately 0.13 cm and approximately 1.5 cm from the first end 141. In yet other embodiments, the position 144 may be any point along the length 143 between the first and second ends 141 and 142. Similar to the first material 121, the second material 131 may be any porous material suitable for use in horizontal flow or lateral flow assay devices that allows for at least (a) binding of the second immobilized molecule 132 thereto and (b) capillary action and transport of non-immobilized liquid (such as the sample 106 and/or the label solution 104 for example). In the embodiment shown in FIGS. 1 and 2, the second material 131 is the HF180 nitrocellulose membrane manufactured by Millipore™. In other embodiments, the second material 131 may be other nitrocellulose membranes that are suitable for use in lateral flow assays, such the Millipore™ HF135 or HF120, Sartorius™ CN140 Membrane, ThermoFisher™ 88018, or Nupore™ FTCN-SH09 for example.

In the embodiment shown in FIGS. 1 and 2, where the second material 131 comprises the HF180 nitrocellulose membrane from Millipore™, the length 143 is approximately 1.5 cm, and the position 144 is approximately 0.75 cm from the first end 141, the sample 106 and/or the label solution 104 may to travel to the position 144 within approximately 1.5 minutes of application to the deposit zone 123 and may travel to the second end 142 within approximately 2 minutes of application to the deposit zone 123. The length 143, the position 144, and the material of the second material 131 may be selected, in combination with other components of the assay device 102, such that the sample 106 and/or the label solution 104 travels to the position 144 between approximately 45 seconds and approximately 5.5 minutes of application to the deposit zone 123 and travels to the second end 142 between approximately 1 minute and approximately 6 minutes of application to the deposit zone 123.

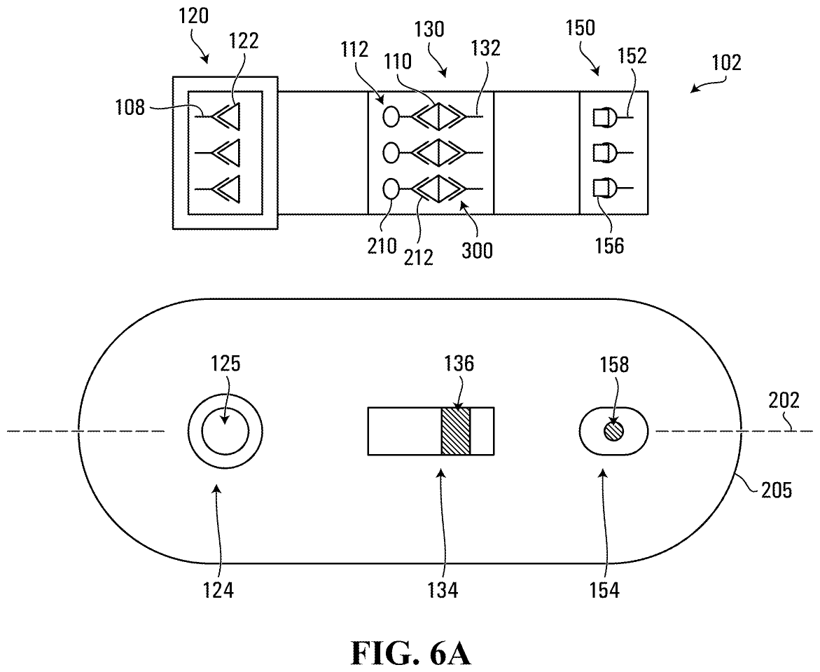

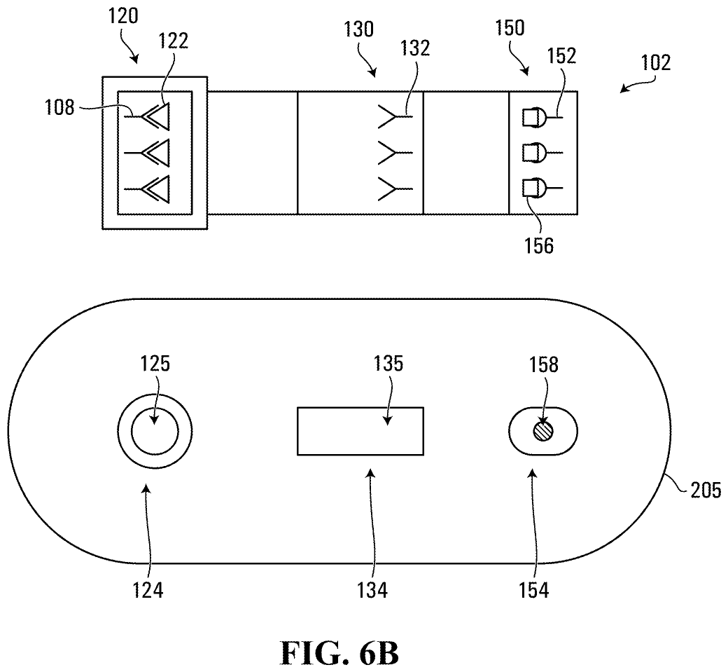

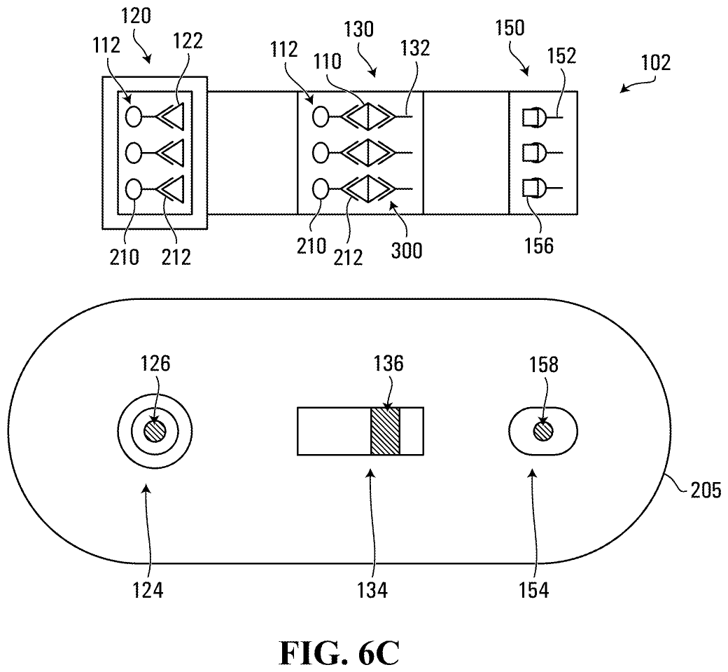

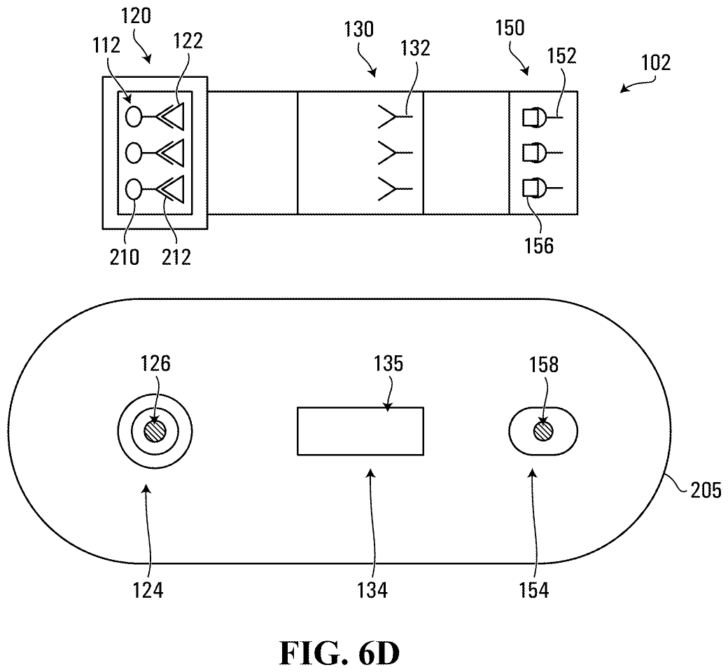

In the embodiment shown in FIGS. 1 and 4, the second immobilized molecule 132 is designed to be capable of specifically binding to the second analyte 110 in the sample 106 to generate a complex 300 (shown in FIGS. 4, 6A and 6C), but not of specifically binding to the binding moiety 212 of the detection molecule 112.

For example, in the embodiment shown in FIG. 1, when the second analyte 110 to be detected in the sample 106 is a naturally occurring antigen from a virus, a bacteria or another microorganism, the second immobilized molecule 132 may be a recombinant or synthetically derived antibody designed to be capable of binding to that antigen analyte 110, while the binding moiety 212 of the detection molecule 112 is a second recombinant or synthetically derived antibody designed to be capable of specifically binding to the complex 300 of the antigen analyte 110-immobilized recombinant antibody 132, but not directly to the immobilized recombinant antibody 132 itself. As a more specific example, in embodiments where the assay system 100 is designed to indicate the presence or absence of a SARS-CoV-2 infection in a host and antibodies produced in response by the immune system of the host, the second immobilized molecule 132 may be a recombinant anti-SARS-CoV-2 N-protein antibody (SEQ ID NO: 3) to capture a SARS-CoV-2 N-protein antigen analyte 110 (or a portion thereof) in the sample 106. As described in greater detail below, the binding moiety 212 may be a recombinant ACE 2 protein (SEQ ID NO: 1) which attaches to a SARS-CoV-2 S-protein antigen analyte 106. As both the SARS-CoV-2 S-protein and N-protein are structural proteins of SARS-CoV-2 and are expressed at the same time, certain SARS-CoV-2 viral particles include both the S-protein and the N-protein. When such SARS-CoV-2 viral particles including both the S-protein and the N-protein specifically bind to the immobilized recombinant anti-SARS-CoV-2 N-protein antibody 132 via its the N-protein portion, the recombinant ACE 2 binding moiety 212 binds to the captured SARS-CoV-2 viral particle via its S-protein portion. Alternatively, as another specific example, in embodiments where the assay system 100 is designed to indicate the presence or absence of an HIV infection in a host and antibodies produced in response by the immune system of the host, the second immobilized molecule 132 may be a recombinant anti-HIV capsid protein antibody (such as a recombinant anti-p24 antibody for example) to capture a HIV capsid protein analyte 110 (such as p24 or portion thereof). As described in greater detail below, the binding moiety 212 may be a recombinant anti-HIV antibody which has dual specificity for both at least one envelope protein (such as gp41 from HIV-1) and at least one capsid protein of the HIV virus (such as capsid protein p24 for example), and both the immobilized recombinant anti-HIV capsid protein antibody 132 and the binding moiety 212 may bind to the HIV capsid protein analyte 110.

Alternatively, in the embodiment shown in FIG. 4, when the second analyte 110′ to be detected in the sample 106 is instead a naturally occurring neutralizing antibody capable of binding to an antigen from a virus, a bacteria, or another microorganism, the second immobilized molecule 132′ may instead be a recombinant or synthetically derived RBD protein which is an analog or a homolog of that antigen, while the binding moiety 212′ of the detection molecule 112 may instead be a recombinant or synthetically derived RBD protein design to be capable of specifically binding to a complex 300′ of the neutralizing antibody analyte 110′-immobilized recombinant RBD protein 132′, but not directly to the immobilized recombinant RBD protein 132′ itself.

The second immobilized molecule 132 and the detection molecule 112 are thus designed to be capable of specifically binding to the second analyte 110 simultaneously and to sandwich the second analyte 110 therebetween.

Similar to the first immobilized molecule 122, the second immobilized molecule 132 may be bound to the second material 131 in a variety of different ways known in the art, and may be bound to the second material 131 via covalent or non-covalent bonds (such as hydrophobic or electrostatic interactions, for example). For example, in embodiments where the second immobilized molecule 132 comprises a recombinant or synthetically derived antibody (shown in FIG. 1) or a recombinant or synthetically derived RBD protein (shown in FIG. 4), the second material 131 may also include a surfactant which partially denatures the amino acids or the folding structure of the second immobilized molecule 132 to encourage hydrophobic or electrostatic interactions between the amino acids of the second immobilized molecule 132 and the fibers of the second material 131.

In certain embodiments (not shown), the immobilized molecule 122 and 132 in the first and second detection regions 120 and 130 may be reversed, such that the first immobilized molecule 122 is bound to the second material 131 in the second detection region 130 and the second immobilized molecule 132 is bound to the first material 121 in the first detection region 120. In such embodiments, the competitive binding assay may occur in the second detection region 130 and the sandwich binding assay may occur in the first detection region 120.

For example, where the second analyte 110 to be detected in the sample 106 is the naturally occurring antigen from a virus, a bacteria or another microorganism and the first analyte 108 to be detected in the sample 106 is a naturally occurring neutralizing antibody capable of binding to that antigen or another antigen from the same virus, bacteria or other microorganism, the first immobilized molecule 122 is the recombinant RBD protein which is an analog or a homolog of the antigen to be bound by the neutralizing antibody analyte 108 and is immobilized in the second detection region 130, the second immobilized molecule 132 is the recombinant antibody designed to be capable of binding to the antigen analyte 110 and is immobilized in the first detection region 120, and the detection molecule 112 includes the binding moiety 212 comprising the recombinant antibody designed to be capable of binding to the immobilized recombinant RBD protein 122 and the complex 300 of the antigen analyte 110-immobilized recombinant antibody 132. The detection molecule 112 and the neutralizing antibody analyte 108 competes to bind to the immobilized recombinant RBD protein 122 in the second detection region 130 to provide an indication of the presence or the absence of the neutralizing antibody analyte 108 in the second detection region 130, while the detection molecule 112 and the immobilized recombinant antibody 132 simultaneously sandwich bind the antigen analyte 110 in the first detection region 120 to provide an indication of the presence or the absence of the antigen analyte 110 in the first detection region 120. Similarly, where the first analyte 108′ to be detected in the sample 106 is the naturally occurring antigen from a virus, a bacteria or another microorganism and the second analyte 110′ to be detected in the sample 106 is a naturally occurring neutralizing antibody capable of binding to the same antigen, or another antigen from the same or different virus, bacteria or other microorganism, the first immobilized molecule 122′ may be the recombinant antibody designed to be capable of binding to the antigen analyte 108′ and is immobilized in the second detection region 130, and the second immobilized molecule 132′ may be the recombinant RBD protein which is an analog or a homolog of the antigen to be bound by the neutralizing antibody analyte 110′ and is immobilized in the first detection region 120, and the detection molecule 112′ includes the binding moiety 212′ comprising the recombinant RBD protein designed to be capable of binding to the immobilized recombinant antibody 122′ and the complex 300′ of the neutralizing antibody analyte 110′-immobilized recombinant RBD protein 132′. The detection molecule 112′ and the antigen analyte 108′ competes to bind to the immobilized recombinant antibody 122′ in the second detection region 130 to provide an indication of the presence or absence of the antigen analyte 108′ in the second detection region 130, while the detection molecule 112′ and the immobilized recombinant RBD protein 132′ simultaneously sandwich bind the neutralizing antibody analyte 110′ in the first detection region 120 to provide an indication of the presence or absence of the neutralizing antibody analyte 110′ in the first detection region 120.

Once the sample 106 and/or the label solution 104 flows in the horizontal direction from the first end 141 to the second end 142 of the second material 131 in the second portion 105, the sample 106 and/or the label solution 104 continues to flow from the second portion 105 in the horizontal direction substantially parallel to the longitudinal axes 202 to the control portion 107 of the assay device 102. The control portion 107 includes a control region 150 configured to receive the sample 106 and/or the label solution 104 from the second portion 105 in the horizontal direction substantially parallel to the longitudinal axis 202. As described above, certain embodiments of the assay device 102 may not include the control portion 107.

The control region 150 includes a control material 151 to which a control molecule 152 is bound. Referring to FIG. 2, the control material 151 has a first end 161, a second end 162, and a length 163 representing a distance between the first end 161 and the second end 162. The control molecule 152 may be bound to the control material 151 at a position 164 along the length 163. Similar to the position 144 of the second immobilized molecule 132, the position 164 of the control molecule 152 may be at a midway point along the length 163. For example, in the embodiment shown in FIG. 2, the length 163 is approximately 2 cm and the position 164 is approximately 1 cm from the first end 161. In other embodiments, the length 163 may range between approximately 0.5 cm and approximately 5 cm and the position 164 may correspondingly range between approximately 0.25 cm and approximately 2.5 cm from first end 161. In other embodiments, the position 164 may be any point along the length 163 between the first and second ends 161 and 162. Similar to the first and second materials 121 and 131, the control material 151 may also be any porous material suitable for use in horizontal flow or lateral flow assays that allows for at least (a) binding of the control molecule 152 thereto and (b) capillary action and transport of non-immobilized liquid (such as the sample 106 and/or the label solution 104 for example). In the embodiment shown in FIGS. 1 and 2, the control material 151 is the HF180 nitrocellulose membrane manufactured by Millipore™. In other embodiments, the control material 151 may be another nitrocellulose membrane suitable for use in lateral flow assays, such as the Millipore™ HF135 or HF120, Sartorius™ CN140 Membrane, ThermoFisher™ 88018, or Nupore™ FTCN-SH09 for example.

In the embodiment shown in FIGS. 1 and 2, where the control material 151 comprises the HF180 nitrocellulose membrane from Millipore™, the length 163 is approximately 2 cm, and the position 164 is approximately 1 cm from the first end 161, the sample 106 and/or the label solution 104 may travel to the position 164 within approximately 14 minutes of application to the deposit zone 123 and may travel to the second end 162 within approximately 16 minutes of application to the deposit zone 123. The length 163, the position 164, and the type of the control material 151 may be selected, in combination with other components of the assay device 102, such that the sample 106 and/or the label solution 104 travels to the position 164 between approximately 7 minutes and approximately 20 minutes of application to the deposit zone 123, and travels to the second end 162 between approximately 8 minutes and approximately 23 minutes of application to the deposit zone 123.

The control molecule 152 is designed to provide an indication that an adequate amount of time has passed for the sample 106 and/or the label solution 104 to flow through the first detection region 120 in the first portion 103 and the second detection region 130 in the second portion 105. In certain embodiments, the control molecule 152 is designed to be capable of specifically binding to a complementary control molecule 156 within the sample 106 and/or a complementary control molecule 157 within the label solution 104. The complementary control molecule 156 may be any molecule found in the sample 106, such as common antigens or antibodies in saliva including protein A or immunoglobulin G for example. The complementary control molecule 156 in the sample 106 may also be a component of a sample buffer 111 of the sample 106 separate from the first and second analytes 108 and 110, such as common buffer components including water, borate, or phosphate for example. The complementary control molecule 157 in the label solution 104 may be the detection molecule 112 and/or a component of a label buffer 113 separate from the detection molecule 112. In the embodiment shown in FIGS. 1 and 6A-6D, the control molecule 152 comprises a recombinant or synthetically derived anti-protein A antibody capable of binding to a protein A complementary control molecule 156 in the sample 106. The control molecule 152 further includes a control label moiety 158 attached thereto. Once the recombinant antibody control molecule 152 binds to the protein A complementary control molecule 156, the control label moiety 158 is released to produce a detectable signal 158 in the control region 150. In other embodiments (not shown), the control molecule 152 may comprise a chemical indicator that changes color when liquid (such as water in the sample 106, and/or the label solution 104) comes into contact with the chemical indicator. For example, the control molecule 152 may comprise sodium hydroxide, phenolphthalein, iodine or copper. In yet other embodiments (not shown), the control molecule 152 may instead comprise a recombinant or synthetically derived RBD protein also capable of binding to the detection molecule 112 in the label solution 104 and having the control label moiety 158 attached thereto.

Similar to the first and second immobilized molecules 122 and 132, the control molecule 152 may be bound to the control material 151 using a variety of different methods known in the art, and may be bound to the control material 151 via covalent and non-covalent bonds (such as hydrophobic or electrostatic interaction for example). For example, in embodiments where the control molecule 152 comprises a recombinant or synthetically derived antibody (shown in FIG. 1) or a recombinant or synthetically derived RBD protein (shown in FIG. 4), the control material 151 may also include a surfactant which partially denatures the amino acids or the folding structure of the control molecule 152 to encourage hydrophobic or electrostatic interactions between the amino acids of the immobilized control molecule 152 and the fibers of the control material 151.

In other embodiments, additional control portions similar to the control portion 107 may be located at various locations in the assay device 102. For example, a second control portion (not shown) may be located in the first portion 103 immediately downstream from, adjacent to, or within, the first detection region 120 to provide an indication that an adequate amount of time has passed for the sample 106 and/or the label solution 104 to flow through the first detection region 120; and/or located in the second portion 105 immediately downstream from, adjacent to, or within, the second detection region 130 to provide an indication that an adequate amount of time has passed for the sample 106 and/or the label solution 104 to flow through the second detection region 130; or any combination thereof.

In certain embodiments, the assay device 102 also includes one or more absorbers and one or more spacers. The spacers are configured to transfer non-immobilized liquid (such as the sample 106 and/or the label solution 104) between detection regions and may also assist in absorbing liquid received by the material of a detection region (such as the first, second and control materials 121, 131 and 151) away therefrom to promote clearer indications of the presence or the absence of the analytes in the detection regions. As described in greater detail below, the material and dimensions of the absorbers and spacers may be selected, in combination with other components of the assay device 102, such that the sample 106 and/or the label solution 104 (having the respective viscosity) travels to the position 164 of the immobilized control molecules 152 within a set amount of time of application to the deposit zone 123.

In the embodiment shown in FIGS. 1 and 2, the assay device 102 includes an absorber 200 in the first portion 103 positioned to absorb liquid received by the first material 121 via the deposit zone 123 and to transfer the liquid from the first material 121 to the second material 131. The assay device 102 also includes a first spacer 180 positioned between the first and second materials 121 and 131 and a second spacer 190 positioned between the second and control materials 131 and 151. In other embodiments, the assay device 102 may include fewer or additional absorbers and spacers. For example, the assay device 102 may only include the first spacer 180, may only include the second spacer 190, or may include additional absorbers positioned under the second material 131 or under the control material 151.

The absorber 200 includes a top surface supporting and in liquid communication with the first material 121 and a distal end 204 overlapping and in liquid communication with a first end 181 of the first spacer 180. In the embodiment shown, the overlap between the distal end 204 of the absorber 200 and the first end 181 of the first spacer 180 is approximately 0.2 cm. In other embodiments, the overlap between the distal end 204 and the first end 181 may range between approximately 0.1 cm and approximately 0.4 cm. In yet other embodiments, the distal end 204 and the first end 181 may be in contact and adjacent, but not overlapping. The absorber 200 is positioned to receive any liquid from the first material 121 in the vertical direction substantially perpendicular to the longitudinal axis 202 and to transfer the liquid in the horizontal direction substantially parallel to the longitudinal axis 202 to the first spacer 180. In the embodiment shown, there is no direct contact between the first material 121 and the first spacer 180. Any of the sample 106 and/or the label solution 104 applied to the first material 121 flows first to the absorber 200 and then to the first spacer 180. This lack of direct contact between the first material 121 and the first spacer 180 may reduce the likelihood of contaminants introduced to the deposit zone 123 or excess molecules on the first material 121 from flowing directly into the first spacer 180 and the second material 131.

The absorber 200 has a height 201 and a length 203 generally defining a volume of the absorber 200. The height 201 and the length 203 may be selected, in combination with other components of the assay device 102, such that the sample 106 and/or the label solution 104 (having the respective viscosity) travels from the first material 121, via the absorber 200, to the first spacer 180 within a set amount of time after application to the deposit zone 123. For example, in the embodiment shown in FIG. 2, the height 201 is approximately 0.5 cm and the length 203 is approximately 2 cm, which may allow the sample 106 and/or the label solution 104 to travel to the first spacer 180 within a few seconds of application to the deposit zone 123. In other embodiments, the height 201 may range between approximately 0.25 cm and approximately 3 cm and the length 203 may range between approximately 1 cm and approximately 5 cm, which may allow the sample 106 and/or the label solution 104 to travel to the first spacer 180, via the absorber 200, between approximately 1 sec and approximately 60 secs of application to the deposit zone 123.

The first spacer 180 is configured to transfer liquid from the first detection region 120 in the horizontal direction substantially parallel to the longitudinal axis 202 to the second detection region 130. Referring to FIG. 2, the first spacer 180 has the first end 181, a second end 182, a length 183 representing a distance between the first end 181 and the second end 182, and a height 184. As described above, the first end 181 overlaps, and is in liquid communication with, the distal end 204 of the absorber 200 and the second end 182 overlaps, and is in liquid communication with, the first end 141 of the second material 131. In the embodiment shown, the overlap between the second end 182 of the first spacer 180 and the first end 141 of the second material 131 is approximately 0.2 cm. In other embodiments, the overlap between the second end 182 and the first end 141 may range between approximately 0.1 cm and approximately 0.4 cm. The length 183 and the height 184 may be selected, in combination with other components of the assay device 102, such that the sample 106 and/or the label solution 104 (having the respective viscosity) travels, via capillary action, to the second end 182 within a set amount of time after application to the deposit zone 123. For example, in the embodiment shown in FIG. 2, the length 183 is approximately 2 cm and the height 184 may be approximately 0.2 cm, which may allow the sample 106 and/or the label solution 104 to travel to the second end 182 within approximately 1 minute of application to the deposit zone 123. In other embodiments, the length 183 may range between approximately 1 cm and approximately 5 cm and the height 184 may range between approximately 0.1 cm and 0.5 cm, which may allow the sample 106 and/or the label solution 104 to travel to the second end 182 between approximately 0.5 min and approximately 3 min of application to the deposit zone 123.

The second spacer 190 is configured to transfer liquid from the second detection region 130 in the horizontal direction substantially parallel to the longitudinal axis 202 to the control region 150. Similar to the first spacer 180, the second spacer 190 has a first end 191, a second end 192, a length 193 representing a distance between the first end 191 and the second end 192, and a height 194. The first end 191 overlaps, and is in liquid communication with, the second end 142 of second material 131 and the second end 192 overlaps, and is in liquid communication with, the first end 161 of the control material 151. In the embodiment shown, the overlap between the first end 191 of the second spacer 192 and the second end 142 of the second material 131 is approximately 0.2 cm, and the overlap between the second end 192 of the second spacer 190 and the first end 161 of the control material 151 is also approximately 0.2 cm. However, in other embodiments, the overlap between the first end 191 and the second end 142 and between the second end 192 and the first end 161 may each range between approximately 0.1 cm and approximately 0.4 cm. Similar to the length 183 and height 184 of the first spacer 180, the length 193 and the height 194 of the second spacer 190 may also be selected, in combination with other components of the assay device 102, such that the sample 106 and/or the label solution 104 (having the respective viscosity) travels to the second end 192 within a set amount of time after application to the deposit zone 123. For example, in the embodiment shown in FIG. 2, the length 193 may be approximately 3 cm and the height 194 may be approximately 0.5 cm, which may allow the sample 106 and/or the label solution 104 to travel to the second end 192 within approximately 12 minutes of application to the deposit zone 123. In other embodiments, the length 193 may range between approximately 1 cm and approximately 5 cm and the height 194 may range between approximately 0.1 cm and 1 cm, which may allow the sample 106 and/or the label solution 104 to travel to the second end 192 between approximately 6 minutes and approximately 17 minutes of application to the deposit zone 123.

The absorber 200 may be any porous material suitable for use in flow-through or vertical flow assays or in horizontal flow or lateral flow assays that allows for absorption, capillary action and transport of non-immobilized liquids (such as the sample 106 and/or the label solution 104 for example). In some embodiments, the absorber 200 may be a polyester membrane. In the embodiment shown in FIGS. 1 and 2, the absorber 200 is bonded fiber. Similarly, the first spacer 180 and the second spacer 190 may be any porous material suitable for use in horizontal flow or lateral flow assays that allows for absorption, capillary action and transport of non-immobilized liquids (such as the sample 106 and/or the label solution 104 for example). In the embodiment shown in FIGS. 1 and 2, the first spacer 180 comprises non-woven cloth and the second spacer 190 comprises fiber glass cloth. In other embodiments, the first spacer 180 and the second spacer 190 may be a polyester membrane. The material of the absorber 200 may be selected, in combination with other components of the assay device 102, such that the sample 106 and/or the label solution 104 travels from the first material 121 to the first spacer 180 within the set amount of time after application to the deposit zone 123. Similarly, the material of the first spacer 180 may also be selected, in combination with other components of the assay device 102, such that the sample 106 and/or the label solution 104 travels to the second end 182 within the set amount of time after application to the deposit zone 123. Similarly again, the material of the second spacer 190 may also be selected, in combination with other components of the assay device 102, such that the sample 106 and/or the label solution 104 travels to the second end 192 within the set amount time after application to the deposit zone 123. Generally, selecting materials such as bonded fiber, non-woven cloth, fiber glass cloth or polytetrafluoroethylene (PTFE) membrane provides slower capillary rate flow times. Selecting materials such as high density polymeric fibers, or polyolefins, such as low-density polyethylene (LDPE), linear low-density polyethylene (LLDPE), high-density polyethylene (HDPE), or polypropylene (PP) provides faster capillary rate flow times. Collectively, the material and dimensions (including the height and length 201 and 203) of the absorber 200, the material and dimensions of the (including the height and length 184 and 183) of the first spacer 180 and material dimensions of the (including the height and length 194 and 193) of the second spacer 190, in combination with the material and dimensions of the other components of the assay device 102, may be selected such that the sample 106 and/or the label solution 104 flows to the second end 192 of the second spacer 190 between approximately 6 minutes and approximately 17 minutes of application to the deposit zone 123.

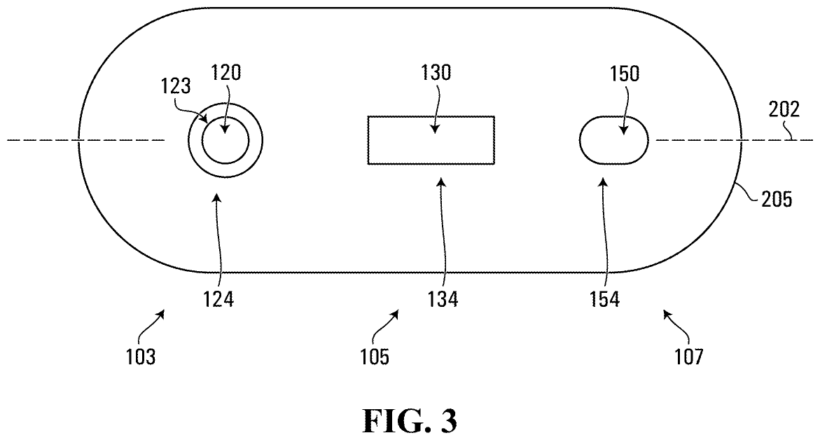

Referring to FIGS. 2 and 3, the assay device 102 may be housed in a housing 205 that provides support for the overall structure of the assay device 102. In the embodiment shown, the housing 205 includes a first window 124 positioned above the first detection region 120 for spatial access to the first detection region 120 to allow the sample 106 and the label solution 104 to be deposited in the deposit zone 123 and visual access to the first detection region 120. The housing 205 also includes a second window 134 positioned above the second detection region 130 for visual access to the second detection region 130, and a control window 154 above the control portion 150 for visual access to the control portion 150. The housing 205 can be made of plastic, glass, or other rigid material to support and house the overall structure of the assay device 102. The housing 205 can also include a handle, markings, or other projections or features that can serve to display information, assist in handling, enable the device to lay flat on a horizontal surface, and the like.

The assay system 100 also includes the label solution 104 comprising the detection molecule 112 suspended in the label buffer 113. Referring to FIGS. 1 and 4, the detection molecule 112 includes the label moiety 210 bound to the binding moiety 212.

The detection molecule 112 functions to indicate the presence or the absence of both a first analyte 108 and a second analyte 110 with a single type of the binding moiety 212. In the embodiment shown, the binding moiety 212 is designed to be capable of specifically binding to the first immobilized molecule 122 in the first detection region 120 and competes with the first analyte 108 for binding to the first immobilized molecule 122 in samples 106 where the first analyte 108 is present. The binding moiety 212 is also designed to be capable of specifically binding to a complex 300 of the second analyte 110-second immobilized molecule 132 in the second detection region 130 in samples 106 where the second analyte 110 is present. The binding moiety 212 may not be capable of binding directly to the second immobilized molecule 132 itself.

In the embodiment shown in FIG. 1, when the second analyte 110 to be detected in the sample 106 is the naturally occurring antigen from a virus, a bacteria or another microorganism and the first analyte 108 to be detected in the sample 106 is a naturally occurring neutralizing antibody capable of binding to that antigen or another antigen from the same or different virus, bacteria, or other microorganism, the first immobilized molecule 122 is a recombinant RBD protein which is an analog or a homolog of the antigen that the neutralizing antibody analyte 108 specifically binds to and the second immobilized molecule 132 is a recombinant antibody designed to be capable of binding to the antigen analyte 110, the binding moiety 212 is a recombinant or synthetically derived antibody designed to be capable of specifically binding to the immobilized recombinant RBD protein 122 in the first detection region 120 and of specifically binding to the complex 300 of the antigen analyte 110-immobilized recombinant antibody 132 in the second detection region 130.

As a more specific example, the assay system 100 may be designed to indicate the presence or absence of SARS-CoV-2 infection in a host and antibodies produced in response by the immune system of the host. As described above, the SARS-CoV-2 virus targets and binds to a human angiotensin converting enzyme 2 (hACE 2) protein found on the surface of specific human cells. In such embodiments, the binding moiety 212 may be a recombinant ACE 2 protein (SEQ ID NO: 1), the first analyte 108 may be an anti-SARS-CoV-2 neutralizing antibody from the sample 106, the second analyte 110 may be a SARS-CoV-2 viral particle (or portion thereof) from the sample 106, the first immobilized molecule 122 may be a RBD of the S-protein from SARS-CoV-2 virus (SEQ ID NO: 2) and the second immobilized molecule 132 may be a recombinant anti-SARS-CoV-2 N-protein antibody (SEQ ID NO: 3). The recombinant ACE 2 binding moiety 212 may compete with the anti-SARS-CoV-2 neutralizing antibody analyte 108 to bind with the immobilized recombinant RBD of the S-protein 122 in the first detection region 120. The recombinant ACE 2 binding moiety 212 may cooperate with the immobilized recombinant anti-SARS-CoV-2 N-protein antibody 132 to sandwich the SARS-CoV-2 antigen analyte 110 (including the N-protein portion to bind to the immobilized recombinant anti-SARS-CoV-2 N-protein antibody 132 and the S-protein portion to bind to the recombinant ACE 2 binding moiety 212 for example).

In another more specific example, the assay system 100 may be designed to indicate the presence or absence of HIV infection in a host and antibodies produced in response by the immune system of the host. In such embodiments, the binding moiety 212 may be a recombinant anti-HIV antibody which has dual specificity for both at least one envelope protein of the HIV virus (such as gp36 from HIV-2, gp41 from HIV-1 and gp120 from HIV-0 for example) and at least one capsid protein of the HIV virus (such as capsid protein p24 for example), the first analyte 108 may be an anti-HIV envelope protein neutralizing antibody from the sample 106 (such as an anti-gp36 neutralizing antibody, an anti-gp41 neutralizing antibody or an anti-gp120 neutralizing antibody for example), the second analyte 110 may be the at least one capsid protein of the HIV virus from the sample 106 (the p24 antigen for example), the first immobilized molecule 122 may be a recombinant RBD of the at least one envelope protein from the HIV virus (such as a recombinant RBD of gp36/gp41/gp120 for example) and the second immobilized molecule 132 may be a recombinant anti-HIV capsid protein antibody (such as a recombinant anti-p24 antibody for example). The anti-HIV detection antibody binding moiety 212 having the dual specificity may compete with the anti-HIV envelope protein neutralizing antibody analyte 108 to bind to the immobilized RBD of the HIV envelope protein 122 (such as gp36/gp41/gp120) in the first detection region 120. The anti-HIV detection antibody binding moiety 212 having the dual specificity may cooperate with the immobilized recombinant anti-HIV capsid protein antibody 132 to sandwich the HIV capsid protein analyte 110 (such as p24) in the second detection region 130.

In other embodiments, the assay system 100 may be designed to indicate the presence or absence of other viral infections including hepatitis B virus (HBV), Dengue, influenza A, or influenza B, and antibodies produced in response by the immune system of the host, in a similar manner to the embodiments for SARS-CoV2 and HIV described above.

In the embodiments shown in FIGS. 1, 4 and 6A-6D, the first analyte 108, the second analyte 110, the immobilized first molecule 122, the immobilized second molecule 132 and the binding moiety 212 of the detection molecule 112 specifically bind to each other via protein-protein interaction. However, in other embodiments, the specific binding may involve other binding systems such as effector and receptor molecules, enzymes and enzyme cofactor combinations, complementary peptide sequences, complementary nucleic acid sequences, and the like.

Upon the binding moiety 212 binding with either the first immobilized molecule 122 in the first detection region 120 or with the complex 300 of the second analyte 110-second immobilized molecule 132 in the second detection region 130, the label moiety 210 bound to the binding moiety 212 produces an indication in the respective first detection region 120 or the second detection region 130. The label moiety 210 may be any substance which is capable of (a) binding to the binding moiety 212 and (b) generating an indicator when the binding moiety 212 specifically binds to a complementary molecule. For example, the label moiety 210 may include one or more of a dye particle, a vat dye particle, a colored particle, a colored bead, an enzyme, a substrate, a chromogen, a catalyst, a fluorescent compound, a chemiluminescent compound, a radioactive label, a colloidal metallic particle, a colloidal gold particle, a colloidal non-metallic particle, a stained microorganism, or a colored organic polymer latex particle. In embodiments where the label moiety 210 comprises a dye particle, the dye particle may specifically be an insoluble vat dye particle comprising at least one hydrophobic benzene ring and at least one charged hydrophilic group, such as a negatively charged sulfur atom or a positively charged nitrogen atom for example. For example, in certain embodiments, the label moiety 210 may comprise particles of vat red 41 having the following formula (1), or derivatives thereof:

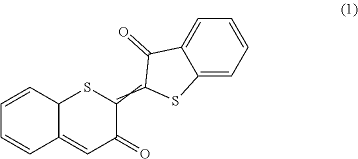 [SMILES: O=C1C=C2C=CC=CC2SC1=C1Sc2ccccc2C1=O]

In other embodiments, the label moiety 210 may instead comprise particles of vat orange 7 having the following formula (2), or derivatives thereof:

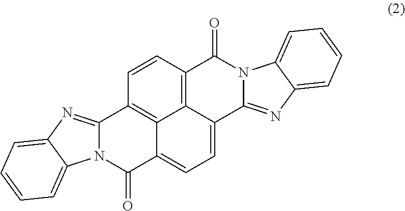 [SMILES: O=c1c2ccc3c4c(ccc(c24)c2nc4ccccc4n12)c(=O)n1c2ccccc2nc31]

In other embodiments, the label moiety 210 may comprise particles of vat red 1 having the following formula (3), or derivatives thereof:

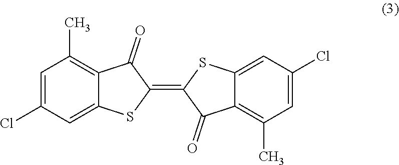 [SMILES: Cc1cc(Cl)cc2c1C(=O)/C(=C1\Sc3cc(Cl)cc(C)c3C1=O)S2]

In other embodiments, the label moiety 210 may comprise particles of istatin having the following formula (4), or derivatives thereof (including N-functionalization, N-arylation, or ring expansions):

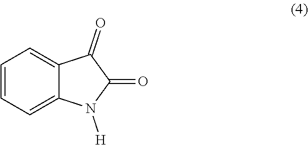 [SMILES: O=C1Nc2ccccc2C1=O]

The label and binding moieties 210 and 212 may be bound together utilizing a variety of different methods known in the art, and may be bound via non-covalent interactions including via hydrophobic and electrostatic interactions for example. Alternatively, the label and binding moieties 210 and 212 may be bound via covalent bonds when the label moiety 210 is another type of label known in the art, such as a fluorescent label (for example, Alexa Fluor™ dyes from ThermoFisher Scientific™). In embodiments where the label moiety 210 comprises the vat dye particle including the at least one charged hydrophilic group (such as the negatively charged sulfur atom or the positively charged nitrogen atom for example) and the binding moiety 212 comprises a polypeptide (such as a recombinant or synthetically derived RBD protein (shown in FIG. 1) or a recombinant or synthetically derived antibody (shown in FIG. 4)), the at least one charged hydrophilic group may facilitate electrostatic interactions with amino acids of the polypeptides having the opposite charge. As more specific examples, the negatively charged sulfur atoms of the vat red 41 molecule (formula (1) above) and the vat red 1 (formula (3) above) may electrostatically interact with, and non-covalently bind to, the positively charged arginine or lysine of the binding moiety 212. Alternatively, the positively charged nitrogen atoms of the vat orange 7 molecule (formula (2) above) and istatin molecule (formula (4) above) may electrostatically interact with, and non-covalently bind to, the negatively charged glutamate or aspartate of the binding moiety 212.

In other embodiments, a pH of the label buffer 113 may be adjusted depending on an isoelectric point of the binding moiety 212 to induce a positive charge or a negative charge in the binding moiety 212 to facilitate binding of the label and binding moieties 210 and 212. For example, where the label moiety 210 comprises particles of vat red 41 (formula (1) above) and the binding moiety 212 comprises a recombinant ACE 2 protein (SEQ ID NO: 1 above), the theoretical isoelectric point for the recombinant ACE 2 protein is 5.36, and the pH of the label buffer 113 may be adjusted to between approximately 5.0 and approximately 5.3 to induce a positive charge in the recombinant ACE 2 binding moiety 212 to facilitate binding to the negatively charged sulfur atoms of the vat red 41 label moiety 210. In certain embodiments, the label buffer 113 may comprise a 0.1 M citrate buffer having citric acid at a concentration of 0.0175 M and sodium citrate dihydrate at a concentration of 0.0825 M, and the pH of the 0.1 M citrate buffer may be adjusted downward with the addition of HCl to the desired pH range of between approximately 5.0 and approximately 5.3. In other embodiments, the label buffer 113 may instead comprise 0.2 M citrate buffer having citric acid at a concentration of 0.0350 M and sodium citrate dihydrate at a concentration of 0.165 M, whereby the pH of the 0.2 M citrate buffer may be adjusted upwards with the addition of NaOH to the desired pH range of between approximately 5.0 and approximately 5.3. In yet other embodiments, the concentration of citric acid and sodium citrate in the label buffer 113 may be adjusted to achieve the desired pH range between approximately 5.0 and approximately 5.3 without any additional acids or bases. For example, the label buffer 113 may include citric acid at a concentration of approximately 0.03698 M and sodium citrate at a concentration of 0.06302 M to arrive at a pH of approximately 5.2. Other types of buffers which are suitable for use as the label buffer 113 for the vat red 41 label moiety 210 and the recombinant ACE 2 binding moiety 212 include buffers including citric acid and Na₂HPO₄, and/or buffers including sodium acetate and acetic acid, for example. Similar to that described above in association with the citrate buffer, refinement and adjustment of the pH of the label buffer 113 may be performed by adding acids or bases to the label buffer 113, such as separately adding NaOH or HCl for example.

In embodiments where the label moiety 210 comprises one or more particles of a vat dye, and the binding moiety 212 comprises a polypeptide (such as the recombinant RBD protein or the recombinant antibody for example), each detection molecule 112 may comprise more than one particle of the vat dye label moiety 210 bound to a single polypeptide binding moiety 212. For example, where the label moiety 210 comprises the particles of vat red 41 (formula (1) above) and the binding moiety 212 comprises the recombinant ACE 2 protein (SEQ ID NO: 1 above), more than one particle of the vat red 41 label moiety 210 may noncovalently interact with, and bind to, each single recombinant ACE 2 binding moiety 212. In other embodiments, the detection molecule 112 may include only a single particle of the label moiety 210 bound to a single protein forming the binding moiety 212.

Particles of the label moiety 210 may have a tendency to aggregate together to form a large aggregation of the label moiety 210. This may be undesirable due to a tendency of such large aggregations to precipitate out of the label buffer 113 and due to the decreased likelihood that such aggregations will noncovalently interact with, and bind to, the binding moiety 212 to form the detection molecule 112. In certain embodiments, the aggregations or particles of the label moiety 210 may be pre-treated to select aggregations or particles that are less than a threshold size to facilitate binding with the binding moiety 212 and to facilitate suspension and/or solubility of the label moiety 210 in the label solution 104. For example, in embodiments where the label moiety 210 comprises one or more particles of vat red 41 (formula (1) above) and the binding moiety 212 comprises the recombinant ACE 2 protein (SEQ ID NO: 1 above), the vat red 41 label moiety 210 may be preselected for aggregations or particles which are between approximately 50 nm and approximately 800 nm using a combination of a sonication step to de-aggregate larger aggregations of the vat red 41 label moiety 210 and a centrifugation step to precipitate out any remaining larger aggregations of the vat red 41 label moiety 210 while leaving smaller aggregations or individual particles of the vat red 41 label moiety 210 suspended in solution. The sonication step may involve a continuous sonication at 100% of approximately 20 kHz for approximately 5 minutes. In other embodiments, the sonication step may involve sonication at between 50% and 100% of approximately 20 kHz for anywhere between approximately 1 minute and approximately 30 minutes. In yet other embodiments, the sonication step may involve pulsed sonication including a cycle of 1 seconds on at 100% of approximately 20 kHz, and 1 second off, for a total time of approximately 10 minutes. The centrifugation step may involve continuous centrifugation at approximately 6000 RPM for approximately 10 minutes. In other embodiments, the centrifugation step may involve centrifugation between approximately 4000 rpm and approximately 10,000 RPM for anywhere between approximately 5 minutes and approximately 30 minutes. After the sonication and centrifugation steps, the average aggregations or particles of vat red 41 label moiety 210 remaining suspended or solubilized in the label buffer 113 may range between approximately 50 nm and 800 nm. In other embodiments, the label buffer 113 containing the label moiety 210 may instead be passed through a 200 nm filter to filter out aggregation of the label moiety 210 which are larger than approximately 200 nm. The particles of the label moiety 210 still remaining suspended or solubilized in the label buffer 113 after the centrifugation step or after the filtration step may be used for subsequent binding to the binding moiety 212.

The label buffer 113 may also include components capable of stabilizing the detection molecule 112 in solution and preventing precipitation of the detection molecule 112 or the label moiety 210 of the detection molecule. Such components may be stabilizing and thickening agents such as glycerol, glycols, glycerin, hyaluronic acid, gelatin, etc., which may increase the viscosity of the label buffer 113 to above a viscosity threshold. For example, in embodiments where the detection molecule 112 includes a label moiety 210 comprising one or more particles of vat red 41 (formula (1) above) and a binding moiety 212 comprising the recombinant ACE 2 protein (SEQ ID (1) above), the label buffer 113 may comprise approximately 10% w/v glycerol. In other embodiments, the label buffer 113 may comprise between approximately 2% w/v glycerol and approximately 15% w/v glycerol.

Additionally, the label buffer 113 may also include components which prevent precipitation or cohesion of any free label moieties 210 which are not bound to the binding moiety 212, and may prevent any such free label moieties 210 from generating the indicator due to unspecific binding during operation of the assay device 102. Such components may be blocking agents such as bovine serum albumin (BSA), casein, skimmed milk powder, whole serum, or whey protein. In embodiments where aggregations or particles of the free label moieties 210 are larger than particles of the blocking agent, the blocking agents may surround any such free label moieties 210 to block the free label moieties 210 from generating the indicator; in contrast, where aggregations or particles of the free label moieties 210 are smaller than particles of the blocking agent, the blocking agents may absorb the free label moieties 210 into the protein structure of the blocking agents to prevent any such free label moieties 210 from generating the indicator. Whey protein has an average molecular weight of approximately 26.6 kDa (corresponding to an average protein size of approximately 2 nm) while BSA has an average molecular weight of 66.5 kDa (corresponding to an average protein size of approximately 7 nm). In embodiments where the label moiety 210 comprises aggregations or particles of vat red 41 between approximately 50 nm and 800 nm (after the sonication and centrifugation steps or the filtration step as described above), both whey protein and BSA may block the function of any free aggregations or particles of vat red 41 label moiety 210 by surrounding it. Due to the smaller size of whey protein in comparison to BSA, whey protein may surround the vat red 41 label moiety 210 more easily and/or with greater efficiency to block its function. The label buffer 113 may comprise approximately 5% w/v whey protein. In other embodiments, the label buffer 113 may comprise between approximately 1% w/v and approximately 10% w/v whey protein.

As described above, the assay system 100 allows detection of at least a first analyte 108 and a second analyte 110 in a sample 106 using a single type of detection molecule 112 having a binding moiety 210. The binding moiety 210 is capable of competing against one of the first and second analytes 108 and 110 for binding with a first immobilized molecule 122 of the assay system 100 and is capable of sandwich binding to the other of the first and second analytes 108 and 110 in cooperation with the second immobilized molecule 132 of the assay system 100. In this respect, the assay system 100 may be used as an assay to discern the presence or absence of both antigen analytes and antibody analytes. In such assays, the binding moiety 212 may be selected to be a molecule within a host that is targeted and bound by an invading organism (or a homolog or derivative thereof) and which is capable of binding to the one of the first and second analytes 108 and 110 which originate from the invading organism (such as an antigen for example) and competes for binding against the other one of the first and second analytes 108 and 110 produced by the host in an immune response against the invading organism (such as a neutralizing antibody for example). For example, as described above, in embodiments where the assay system 100 is designed to indicate the presence or absence of SARS-CoV-2 infection in a host and the presence or absence of an immune response of the host to the SARS-CoV-2 infection, the binding moiety 212 may be a recombinant ACE 2 protein (SEQ ID NO: 1 above), the first analyte 108 may be an anti-SARS-CoV-2 S-protein neutralizing antibody from the sample 106, the second analyte 110 may be a SARS-CoV-2 viral particle (or portion thereof) including both a S-protein portion and a N-protein portion, from the sample 106, the first immobilized molecule 122 may be a recombinant RBD of the S-protein from SARS-CoV-2 virus (SEQ ID NO: 2 above) and the second immobilized molecule 132 may be a recombinant anti-SARS-CoV-2 N-protein antibody (SEQ ID NO: 3 above). As another example, and as also described above, in embodiments where the assay system 100 is designed to indicate the presence or absence of HIV-1 infection in a host and the presence or absence of an immune response of the host to the HIV-1 infection, the binding moiety 212 may be an anti-HIV antibody which has dual specificity for both gp36 of HIV-1 and p24 of the HIV, the first analyte 108 may be anti-gp36 neutralizing antibody in the sample 106, the second analyte 110 may be the p24 antigen (or a portion thereof) in the sample 106, the first immobilized molecule 122 may be a RBD of the gp36 from the HIV-1 and the second immobilized molecule 132 may be a recombinant anti-p24 antibody.

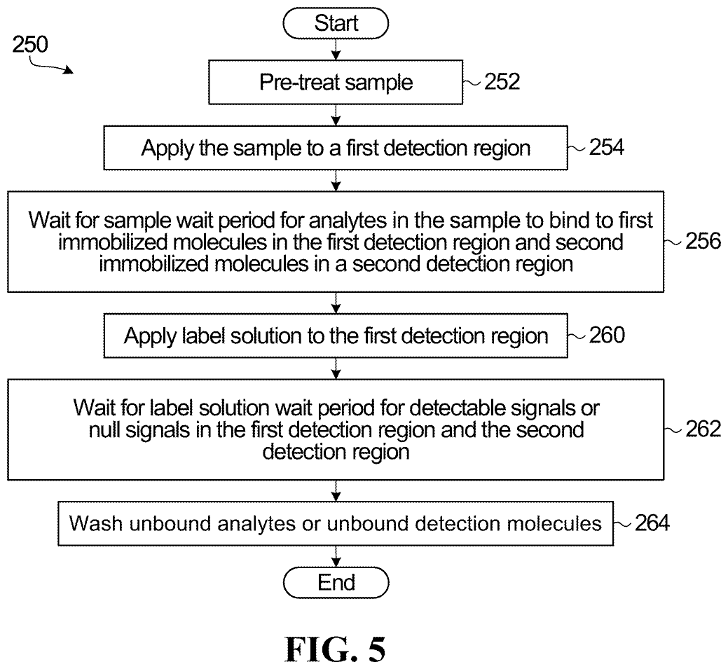

Referring to FIG. 5, a method of detecting the presence or absence of at least a first analyte 108 and a second analyte 110 in a sample 106 using an assay system (such as the assay system 100 shown in FIGS. 1-3) according to one embodiment is shown generally at 250.

The method 250 begins at block 252, which involves pre-treating a sample 106 for use with the assay system 100. Pre-treatment may involve preparing the sample 106 for application to the assay device 102, such as by separating plasma from blood, diluting viscous liquids, adjusting pH of the sample 106, for example, and may also involve filtration, distillation, separation, concentration, inactivation of interfering components, the addition of reagents, or other sample preparation techniques. As a specific example, in embodiments where the sample 106 comprises saliva, the saliva may be diluted in the sample buffer 111 to generate the sample 106.

The method 250 then continues to block 254, which involves applying the pre-treated sample 106 to the first detection region 120 in generally the vertical direction substantially perpendicular to the longitudinal axis 202 of the assay device 102. For example, as described above, the sample 106 may be applied at the first window 124 of the housing 205 onto the deposit zone 123. The method 250 then continues to block 256, which involves waiting for a sample wait period for the sample 106 to permeate the first material 121 in the first detection region 120 and to (or begin to) migrate in the horizontal direction parallel to the longitudinal axis 202 of the assay device 102 to the second material 131 in the second detection region 130 and the control material 151 in the control region 150. Block 256 provides time for the first analytes 108 (if any) in the sample 106 to bind to the first immobilized molecule 122 in the first detection region 120 and the second analytes 110 (if any) in the sample 106 to bind to the second immobilized molecule 132 in the second detection region 130.

In certain embodiments, the sample wait period may be based on sufficient permeation of the sample 106 in the first material 121, and may depend in part on volume of the sample 106, the material of the first material 121, and the material, height 201 and length 203 of the absorber 200. For example, in embodiments where the volume of the sample 106 is approximately 0.5 mL, the first material 121 comprises Millipore™ HF180 nitrocellulose membrane, and the absorber 200 comprises bonded fiber having a height 201 of approximately 0.5 cm and a length 203 of approximately 2 cm, the sample wait period at block 256 may be less than 60 seconds. In other embodiments, the sample wait period may instead be based on migration of the sample 106 to the second end 142 of the second material 131, and may depend further in part on the material, the length 183 and the height 184 of the first spacer 180, and the material and the length 143 of the second material 131. For example, in embodiments where the volume of the sample 106 is approximately 0.5 mL, the first material 121 comprises Millipore™ HF180 nitrocellulose membrane, the absorber 200 comprises bonded fiber and has a height 201 of approximately 0.5 cm and a length 203 of approximately 2 cm, the first spacer 180 comprises a non-woven cloth and has a length 183 of approximately 2 cm and a height 184 of approximately 0.2 cm, and the second material 131 comprises Millipore™ HF180 nitrocellulose membrane and has a length 143 of approximately 1.5 cm, the sample wait period at block 256 may be less than 2 minutes. In yet other embodiments, the sample wait period may instead based on migration of the sample 106 to the position 164 of the immobilized control molecule 152 in the control material 151, and may depend further in part on the material and length 193 of the second spacer 190 and the material and length 163 of the control material 151. For example, in embodiments where the volume of the sample 106 is approximately 0.5 mL, the first material 121 comprises Millipore™ HF180 nitrocellulose membrane, the absorber 200 comprises bonded fiber and has a height 201 of approximately 0.5 cm and a length 203 of approximately 2 cm, the first spacer 180 comprises a non-woven cloth and has a length 183 of approximately 2 cm and a height 184 of approximately 0.2 cm, the second material 131 comprises Millipore™ HF180 nitrocellulose membrane and has a length 143 of approximately 1.5 cm, the second spacer 190 comprises fiber glass cloth and has a length 193 of approximately 3 cm and a height 194 of approximately 0.5 cm, the control material 151 comprises Millipore™ HF180 nitrocellulose membrane and has a length 163 is approximately 2 cm and the position 164 of the immobilized control molecule 152 is approximately 1 cm from the first end 161 of the control material 151, the sample wait period at block 256 may be approximately 14 minutes.

In yet other embodiments, the sample wait period at block 256 may instead be based on a visual indication that the sample 106 has a reached a control portion, such as the control portion 107 shown in FIGS. 1-3 for example. In such embodiments, the immobilized control molecule 152 in the control region 150 may include an indicator designed to bind to a complementary control molecule 156 found in the sample 106, such as common antigens or antibodies including protein A or immunoglobulin G in saliva, or common components from the sample buffer 111 including water or phosphate for example. In the embodiment shown in FIGS. 1-3 and 6A-6D, the control molecule 152 comprises a recombinant anti-protein A protein antibody capable of binding to a protein A complementary control molecule 156 in the sample 106 and having a control label moiety 158 which is released upon the immobilized anti-protein A antibody control molecule 152 binding to the protein A complementary control molecule 156 to generate a visual indicator. Such embodiments may allow an operator of the assay device 102 to wait until the visual indicator is displayed in the control window 154 (shown in FIGS. 2 and 3) instead of waiting any set amount of time at block 256.

The method 250 then continues to block 260, which involves applying the label solution 104 to the first detection region 120 in generally the vertical direction perpendicular to the longitudinal axis 202 of the assay device 102. For example, the label solution 104 may also be applied at the first window 124 onto the deposit zone 123. The method 250 then continues to block 262, which involves waiting for a label solution wait period for the label solution 104 to permeate the first material 121 in the first detection region 120 and to permeate the second material 131 in the second detection region 130. Block 262 provides time for the detection molecule 112 to bind to the first immobilized molecule 122 in the first detection region 120 if any are unbound by the first analytes 108 of the sample 106 after block 256 and/or any complexes 300 of the second analyte 110-second immobilized molecule 132 in the second detection region 130 if any are formed after block 256.

As described above, in the embodiment shown in FIGS. 1-3, the single type of detection molecule 112 allows for detection of both the first analyte 108 via a competitive assay in the first detection region 120 and the second analyte 110 via a sandwich assay in the second detection region 130. Employing a single type of detection molecule for double analyte detection as described in the present application can reduce manufacturing costs, simplify the use of the assay device 102, and provide more informative results by simultaneously displaying indicators of two related analytes. As also described above, the competitive assay in the first detection region 120 involves the detection molecule 112 and the first analyte 108 competing to bind to the first immobilized molecule 122 in the first detection region 120. Due to the competitive relationship between the first analyte 108 and the detection molecule 112 for the first immobilized molecule 122, the detection molecule 112 may block binding of the first analyte 108 if both are simultaneously introduced to the first detection region 120, which can increase the likelihood of a false negative result for the first analyte 108 (an indicator that there is no first analyte 108 in the sample 106 when there is first analyte 108 in the sample 106). However, the likelihood of such false negative result is decreased with the assay system 100 where the label solution 104 is provided as a solution separate from the assay device 102. A separate label solution 104 allows the sample 106 to be applied to the first detection zone 120 at block 254 prior to application of the label solution 104 to the first detection zone 120 at block 260. The staggered application of the sample 106 and the label solution 104 allows binding of the first analyte 108 (if any) in the sample 106 to the first immobilized molecule 122 to be prioritized over binding of the labeled detection molecule 112 to the first immobilized molecule 122. Introducing the detection molecule 112 after the first analyte 108 has already had the opportunity to bind to the first immobilized molecule 122 reduces the likelihood of any false negative results for the first analyte 108.

Referring now to FIG. 6A, in embodiments where the sample 106 contains both the first analyte 108 and the second analyte 110, the first analyte 108 will bind to the first immobilized molecule 122 in the first detection region 120 and the second analyte 110 will bind to the second immobilized molecule 132 in the second detection region 130 to form the complex 300 after block 256. The binding of the first analyte 108 to the first immobilized molecule 122 will prevent the binding moiety 212 of the detection molecule 112 from binding (or limit the amount thereof which can bind) to the first immobilized molecule 122 in the first detection region 120 after block 262. Preventing or limiting binding of the detection molecule 112 to the first immobilized molecule 122 generates a null signal 125 in the first detection region 120, indicating the presence of the first analyte 108 in the sample 106. The null signal 125 in the first detection region 120 may be an absence of a visual indicator generated by the label moiety 210 of the detection molecule 112. The binding of the second analyte 110 to the second immobilized molecule 132 to form the complex 300 after block 256 will allow the binding moiety 212 of the detection molecule 112 to bind to the complexes 300 in the second detection region 130 after block 262. Binding of the detection molecule 112 to the complex 300 generates a detectable signal 136 in the second detection region 130 indicating the presence of the second analyte 110 in the sample 106. The detectable signal 136 in the second detection region 130 may be a presence of the visual indicator generated by the label moiety 210 of the detection module 112. In embodiments where the assay system 100 is designed to indicate the presence or the absence of SARS-CoV-2 infection in a host and neutralizing antibodies produced by the immune system of the host in response, the first analyte 108 may be the anti-SARS-CoV-2 neutralizing antibody from the sample 106 and the second analyte 110 may be the SARS-CoV-2 antigen, utilizing the assay system 100 to determine that both the natural anti-SARS-CoV-2 neutralizing antibody analyte 108 and the SARS-CoV-2 antigen analyte 110 are present in the sample 106 can indicate that the host providing the sample 106 has an active SARS-CoV-2 infection and has been infected for a sufficient amount of time for the host's immune system to generate an antibody response. In embodiments where the assay system 100 is designed to indicate the presence or the absence of HIV-1 infection in a host and neutralizing antibodies produced by the immune system of the host in response, the first analyte 108 may be an anti-HIV-1 neutralizing antibody from the sample 106 and the second analyte 110 may be an HIV-1 antigen (or portion thereof) from the sample 106, utilizing the assay system 100 to determine that both the anti-HIV-1 neutralizing antibody analyte 108 and the HIV-1 antigen analyte 110 are present in the sample 106 can indicate that the host providing the sample 106 has an active HIV-1 infection and a detectable viral load and has been infected for a sufficient amount of time for the host's immune system to generate an antibody response.

Referring now to FIG. 6B, when the sample 106 does contain the first analyte 108 but does not contain the second analyte 110, the first analyte 108 will bind to the first immobilized molecule 122 in the first detection region 120 but no second analyte 110 will bind to the second immobilized molecule 132 in the second detection region 130 after block 256. Similar to that described in association with FIG. 6A above, the binding of the first analyte 108 to the first immobilized molecule 122 after block 256 will prevent or limit the binding of the detection molecule 112 to the first immobilized molecule 122, which generates the null signal 125 in the first detection region 120 after block 262, indicating the presence of the first analyte 108 in the sample 106. In contrast, the lack of binding of the second analyte 110 to the second immobilized molecule 132 after block 256 will prevent or limit the formation of any complexes 300 for the detection molecule 112 to bind to in the second detection region 130 after block 262. When no complexes 300 of the second analyte 110-second immobilized molecule 132 are formed, the detection molecule 112 cannot bind to any such complexes 300, which generates a null signal 135 in the second detection region 130 after block 262, indicating the absence of the second analyte 110 in the sample 106. Similar to the null signal 125 in the first detection region 120, the null signal 135 in the second detection region 130 may be an absence of the visual indicator generated by the label moiety 210 of the detection molecule 112. In embodiments where the first analyte 108 is the anti-SARS-CoV-2 neutralizing antibody and the second analyte 110 is the SARS-CoV-2 antigen, utilizing the assay system 100 to determine that the natural anti-SARS-CoV-2 neutralizing antibody analyte 108 is present but that the SARS-CoV-2 antigen analyte 110 is absent in a sample 106 can indicate that the host providing the sample 106 does not have an active SARS-CoV-2 infection but was previously infected with SARS-CoV-2 or has received a vaccination for SARS-CoV-2. In embodiments where the first analyte 108 is the anti-HIV-1 neutralizing antibody and the second analyte 110 is the HIV-1 antigen, utilizing the assay system 100 to determine that the anti-HIV-1 neutralizing antibody analyte 108 is present but that the HIV-1 antigen analyte 110 is absent in a sample 106 can indicate that the host providing the sample 106 has a HIV-1 infection, but that the viral load of HIV-1 is low.

Referring now to FIG. 6C, when the sample 106 does not contain the first analyte 108 but does contain the second analyte 110, the first analyte 108 will not bind to the first immobilized molecule 122 in the first detection region 120 but the second analyte 110 will bind to the second immobilized molecule 132 to form the complex 300 in the second detection region 130. The lack of binding of the first analyte 108 to the first immobilized molecule 122 after block 256 leaves the first immobilized molecule 122 available for binding to the detection molecule 112 after block 262. Binding of the detection module 112 to the first immobilized molecule 122 generates a detectable signal 126 in the first detection region 120 after block 262, indicating the absence of the first analyte 108 in the sample 106. Similar to the detectable signal 136 in the second detection region 130, the detectable signal 126 in the first detection region 120 may be the presence of the visual indicator generated by the label moiety 210 of the detection molecule 112. Similar to that described above in association with FIG. 6A, binding of the second analyte 110 and the second immobilized molecule 132 to form the complex 300 after block 256 allows the detection molecule 112 to bind to the complex 300 after block 262, which generates the detectable signal 136 in the second detection region 130 after block 262, indicating the presence of the second analyte 110 in the sample 106. In embodiments where the first analyte 108 is the anti-SARS-CoV-2 neutralizing antibody and the second analyte 110 is the SARS-CoV-2 antigen, utilizing the assay system 100 to determine that the natural anti-SARS-CoV-2 neutralizing antibody analyte 108 is absent but that the SARS-CoV-2 antigen analyte 110 is present in a sample 106 can indicate that the host providing the sample 106 has an active SARS-CoV-2 infection but has not been infected for a sufficient amount of time for the host's immune system to generate an antibody response. In embodiments where the first analyte 108 is the anti-HIV-1 neutralizing antibody and the second analyte 110 is the HIV-1 antigen, utilizing the assay system 100 to determine that the anti-HIV-1 neutralizing antibody analyte 108 is absent but that the HIV-1 antigen analyte 110 is present in a sample 106 can indicate that the host providing the sample 106 has an active HIV-1 infection but has not been infected for a sufficient amount of time for the host's immune system to generate an antibody response.

Finally, referring to FIG. 6D, when the sample 106 does not contain the first analyte 108 and does not contain the second analyte 110, no first analyte 108 will bind to the first immobilized molecule 122 in the first detection region 120 and no second analyte 110 will bind to the second immobilized molecule 132 to form the complex 300 in the second detection region 130. Similar to that described above in association with FIG. 6C, the lack of binding of the first analyte 108 to the first immobilized molecule 122 after block 256 leaves the first immobilized molecule 122 available for binding with the detection molecule 112 after block 262, which generates the detectable signal 126 in the first detection region 120 after block 262, indicating the absence of the first analyte 108 in the sample 106. Similar to that described above in association with FIG. 6B, lack of binding of the second analyte 110 and the second immobilized molecule 132 after block 256 means a lack of complexes 300 for the detection molecule 112 to bind to after block 262, which generates the null signal 135 in the second detection region 130 indicating the absence of the second analyte 110 in the sample 106. In embodiments where the first analyte 108 is the anti-SARS-CoV-2 neutralizing antibody and the second analyte 110 is the SARS-CoV-2 antigen, utilizing the assay system 100 to determine that both the anti-SARS-CoV-2 neutralizing antibody analyte 108 and the SARS-CoV-2 antigen analyte 110 is absent in a sample 106 can indicate that the host providing the sample 106 does not have an active SARS-CoV-2 infection, has not been infected with SARS-CoV-2 in the recent past, and/or has not received a vaccine for the SARS-CoV-2 in the recent past. In embodiments where the first analyte 108 is the anti-HIV-1 neutralizing antibody and the second analyte 110 is the HIV-1 antigen (or a portion thereof), utilizing the assay system 100 to determine that both the natural anti-HIV-1 neutralizing antibody analyte 108 and the HIV-1 antigen analyte 110 is absent in a sample 106 can indicate that the host providing the sample 106 does not have an active HIV-1 infection and has not been infected with HIV-1.

Referring back to FIG. 5, the label solution wait period at block 262 may be based on migration of the label solution 104 to the second end 142 of the second material 131, and may depend on in part on volume of the label solution 104, the material of the first material 121, the material, height 201 and length 203 of the absorber 200, the material, length 183 and height 184 of the first spacer 180, and the material and length 143 of the second material 131. For example, in embodiments where the volume of the label solution 104 is approximately 0.5 mL, the first material 121 comprises Millipore™ HF180 nitrocellulose membrane, the absorber 200 comprises bonded fiber and has a height 201 of approximately 0.5 cm and a length 203 of approximately 2 cm, the first spacer 180 comprises non-woven cloth and has a length 183 of approximately 2 cm and a height 184 of approximately 0.2 cm, and the second material 131 comprises the Millipore™ HF180 and has a length 143 of approximately 1.5 cm, the label solution wait period at block 262 may be less than 2 minutes. In other embodiments, the label solution wait period may instead be based on migration of the label solution 104 to the position 164 of the immobilized control molecule 152 in the control material 151, and may depend further in part on the material, the length 193 and the height 194 of the second spacer 190, and the material and length 163 of the control material 151. For example, in embodiments where the volume of the label solution 104 is approximately 0.5 mL, the first material 121 comprises Millipore™ HF180 nitrocellulose membrane, the absorber 200 comprises bonded fiber and has a height 201 of approximately 0.5 cm and a length 203 of approximately 2 cm, the first spacer 180 comprises non-woven cloth and has a length 183 of approximately 2 cm and a height 184 of approximately 0.2 cm, the second material 131 comprises Millipore™ HF180 nitrocellulose membrane and has a length 143 of approximately 1.5 cm, the second spacer 190 comprises fiber glass cloth and has a length 193 of approximately 3 cm and a height 194 of approximately 0.5 cm, the control material 151 comprises Millipore™ HF180 nitrocellulose membrane and has a length 163 of approximately 2 cm, and the position 164 of the immobilized control molecule 152 is approximately 1 cm from the first end 161 of the control material 151, the sample wait period at block 262 may be approximately 14 minutes.

In yet other embodiments, similar to the sample wait period at block 256, the label solution wait period at block 262 may instead be based on a visual indication that the label solution 104 has a reached a control portion, such as the control portion 107 shown in FIGS. 1-3 for example. In such embodiments, the immobilized control molecule 152 in the control region 150 may include an indicator designed to bind to the complementary control molecule 157 in the label solution 104, such as common components from the label buffer 113 including water, to generate a visual indication of when the immobilized control molecule 152 binds to the complementary control molecule 157 in the label solution 104 indicating that the label solution 104 has reached the control region 150. In other embodiments, the immobilized control molecule 152 may instead bind directly to the detection molecule 112 in the label solution 104. In such embodiments, binding of the immobilized control molecule 152 and the detection molecule 112 in the control region 150 may generate the presence signal 156 in the control region 150 indicating that the label solution 104 has reached the control region 150.

The method 250 then continues to optional block 264, which involves applying a wash solution to the first detection region 120 in generally the vertical direction perpendicular to the longitudinal axis 202 of the assay device 102. For example, the wash solution may also be applied in the first window 124 onto the deposit zone 123. Optional block 264 can remove any first analytes 108 or second analytes 110 (or any other components) in the sample 106 which have weakly and non-specifically bound the first immobilized molecule 122 in the first detection region 120 and/or the second immobilized molecule 132 in the second detection region 130, and also remove any detection molecules 212 in the label solution 104 which have only weakly and/or non-specifically bound to the first immobilized molecule 122 in the first detection region 120 or to the complex 300 of the second analyte 110-second immobilized molecule 132 in the second detection region 130. The wash solution used may be phosphate-buffered saline (PBS), tris, or borate buffer, for example, and may include sodium dodecyl sulfate (SDS), Triton™ X-100, and/or or Tween™ 20.

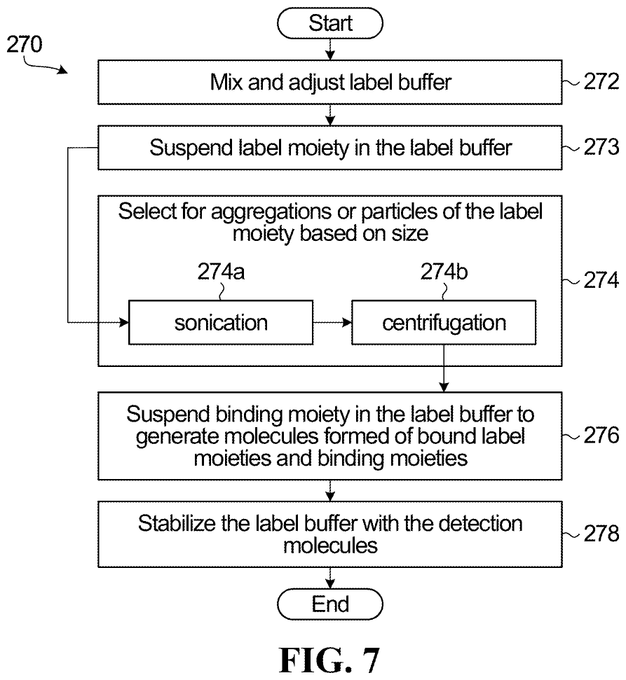

Referring to FIG. 7, a method of preparing the label solution 104 including the detection molecule 112 and the label buffer 113 for use with the assay system 100 is according to one embodiment is shown generally at 270.

The method 270 begins at block 272, which involves mixing and adjusting an appropriate label buffer 113 for use with the desired label moiety 210 and the desired binding moiety 212. The pH of the label buffer 113 may also be adjusted at block 272 depending on the isoelectric point of the desired binding moiety 212 to induce a positive charge or a negative charge in the desired binding moiety 212 to facilitate binding with the desired label moiety 210. For example, as described above, in embodiments where the label moiety 210 to be used is vat red 41 (formula (1) above) and the binding moiety 212 to be used is a recombinant ACE 2 protein (SEQ ID NO: 1 above), the label buffer 113 may be an acidic 0.1 M citrate buffer having citric acid at a concentration of 0.0175 M and sodium citrate dihydrate at a concentration of 0.0825 M. The pH of the 0.1 M citrate buffer may be adjusted downward with the addition of HCl to the desired pH range of between approximately 5.0 and approximately 5.3. Other buffers which are suitable for use with the vat red 41 label moiety 210 and the recombinant ACE 2 binding moiety 212 include other acidic buffer systems such as buffers including citric acid and Na₂HPO₄, and/or buffer systems including sodium acetate and acetic acid.

The method 270 then continues to block 273, which involves suspending a sufficient amount of label moiety 210 in the label buffer 113 buffer to produce an initial label suspension. A sufficient amount of the label moiety 210 may be between approximately 0.1% w/v and approximately 5.0% w/v of label moiety 210 in the label buffer 113, but can depend on identity and size of the label moiety 210. For example, in embodiments where the label moiety 210 comprises vat red 41 (formula (1) above), block 274 involves adding approximately 5 g of the vat red 41 label moiety 210 to approximately 500 mL of the label buffer 113 to achieve a concentration of approximately 1.0% w/v of the vat red 41 label moiety 210. In other embodiments, block 272 may involve mixing the vat red 41 label moiety 210 and the label buffer 113 to reach a concentration anywhere between approximately 0.1% w/v and approximately 1.0% w/v.

The method 270 then continues to block 274, which involves pre-selecting for aggregations or particles of the label moiety 210 based on size to facilitate noncovalent binding of the label moiety 210 to the binding moiety 212. For example, block 274 may involve an initial sonication block 274a to de-aggregate large aggregations of particles of the label moiety 210 and a subsequent centrifugation block 274b to precipitate out larger aggregations of the label moiety 210 while leaving the smaller aggregations or individual particles suspended in the label buffer 113. In embodiments where the label moiety 210 comprises vat red 41 (formula (1) above) and the binding moiety 212 comprises a recombinant ACE 2 protein (SEQ ID NO: 1 above), the sonication block 274a may involve continuous sonication at 100% of approximately 20 kHz for approximately 5 minutes. In other embodiments, the sonication block 274a may involve sonication between 50% and 100% of approximately 20 kHz for anywhere between approximately 1 minute and approximately 30 minutes. In yet other embodiments, the sonication block 274a may involve pulsed sonication having a cycle of 1 seconds on at 100% of approximately 20 kHz, and 1 second off, for a total time of approximately 10 minutes. The centrifugation block 274b may involve continuous centrifugation at approximately 6000 RPM for approximately 10 minutes. In other embodiments, the centrifugation block 274b may involve centrifugation between approximately 4000 rpm and approximately 10,000 RPM for between approximately 5 minutes and approximately 30 minutes. After the sonication and centrifugation blocks 274a and 274b, the average aggregations or particles of vat red 41 label moiety 210 remaining suspended or solubilized in the label buffer 113 may range between approximately 50 nm and 800 nm.

In other embodiments, block 274 may instead involve filtering the label buffer 113 containing the label moiety 210 through a filter to filter out larger aggregations of the label moiety 210. For example, in embodiments where the label moiety 210 comprises vat red 41 (formula (1) above), the label buffer 113 containing the vat red 41 label moiety 210 may instead be passed through a 200 nm filter to filter out aggregations of the label moiety 210 which are larger than approximately 200 nm.

The particles of the label moiety 210 still remaining suspended or solubilized in the label buffer 113 after block 274 may then be collected for subsequent binding to the binding moiety 212.

The method 270 then continues at block 276, which involves suspending the binding moiety 212 in the label buffer 113 including the label moiety 210 to facilitate binding of the label and binding moieties 210 and 212 to form the detection molecule 112. Suspending the binding moiety 212 may involve suspending a sufficient amount of binding moiety 212 to promote binding of the label and binding moieties 210 and 212 in the label buffer 113. A sufficient amount of binding moiety 212 may mean between approximately 1 ug/mL and approximately 10 ug/mL of binding moiety 212 in the label buffer 113, but can depend on the identity and size of the binding moiety 212. For example, in embodiments where the label moiety 210 comprises aggregations or particles of vat red 41 (formula (1) above) and the binding moiety 212 comprises a recombinant ACE 2 protein (SEQ ID NO: 1 above), the recombinant ACE 2 binding moiety 212 may be added to the label buffer 113 including the vat red 41 label moiety 210 to achieve a concentration of approximately 5 ug/mL of the recombinant ACE 2 binding moiety 212. When the label buffer 113 was initially mixed at block 272, the pH of the label buffer 113 may be adjusted depending on the anticipated isoelectric point of the desired binding moiety 212. In embodiments where the label moiety 210 comprises particles of vat red 41 (formula (1) above) and the binding moiety 212 comprises a recombinant ACE 2 protein (SEQ ID NO: 1 above) having the isoelectric point of approximately 5.36, block 272 involved adjusting the pH of the label buffer 113 to a desired range of between approximately 5.0 and approximately 5.3 as described above. Upon the addition of the recombinant ACE 2 binding moiety 212 to the pH-adjusted label buffer 113 at block 276, the pH of the label buffer 113 induces a positive charge in the recombinant ACE 2 binding moiety 212 to facilitate binding to the negatively charged sulfur atoms of the vat red 41 label moiety 210 to form the detection molecule 112.

The method 270 then continues at block 278, which involves stabilizing the detection molecules 112 formed in the label buffer 113 to generate the label solution 104 suitable for use with the assay device 102. A sufficient amount of thickening and blocking agents may be added to the label buffer 113 at block 278. A sufficient amount of thickening agent may be between approximately 2% w/v and approximately 15% w/v of the thickening agent in the label buffer 113, while a sufficient amount of blocking agent may be between approximately 1% w/v and approximately 10% w/v of the blocking agent in the label buffer 113, but both may depend on the size and identity of the blocking agent the thickening agent, as well as the identity of the label and binding moieties 210 and 212. In embodiments where the label moiety 210 comprises particles of vat red 41 molecule (formula (1) above), the binding moiety 212 comprises recombinant ACE 2 protein (SEQ ID NO: 1 above), the thickening agent may comprise glycerol and the blocking agent comprises whey protein. Glycerol may be added to the label buffer 113 to achieve a concentration of approximately 10% w/v, while whey protein may be added to the label buffer 113 to achieve a concentration of approximately 5% w/v. The stabilized label buffer 113 including the detection molecules 112 may then be used as the label solution 104 with the assay device 102.

While specific embodiments have been described and illustrated, such embodiments should be considered illustrative of the subject matter described herein and not as limiting the claims as construed in accordance with the relevant jurisprudence.

1. An assay system for detecting presence or absence of at least a first analyte and a second analyte in a sample, the assay system comprising:
a label solution comprising a detection molecule; and
an assay device comprising:
a first detection region configured to receive the sample in a vertical direction perpendicular to a longitudinal axis of the assay device;
a second detection region in liquid communication with the first detection region and configured to receive the sample from the first detection region in a horizontal direction parallel to the longitudinal axis;
a first immobilized molecule immobilized in one of the first and second detection regions and configured to bind to either the detection molecule or the first analyte to indicate the presence or the absence of the first analyte in the sample; and
a second immobilized molecule immobilized in the other one of the first and second detection regions and configured to bind to the second analyte to generate a complex, wherein the detection molecule is also configured to bind to the complex to indicate the presence or the absence of the second analyte in the sample.

2. The assay system of claim 1, wherein the first immobilized molecule is immobilized in the first detection region and the second immobilized molecule is immobilized in the second detection region.

3. The assay system of claim 2, wherein:
if the first immobilized molecule remains unbound by the first analyte after the sample is applied to the first detection region, the detection molecule binds to the first immobilized molecule to generate a detectable signal indicating the absence of the first analyte in the sample, and
if the first immobilized molecule binds to the first analyte after the sample is applied to the first detection region, the detection molecule does not bind to the first immobilized molecule and generates a null signal indicating the presence of the first analyte in the sample.

4. The assay system of claim 2, wherein:
if the second immobilized molecule binds to the second analyte to generate the complex after the sample is applied to the second detection region, the detection molecule binds to the complex to generate a detectable signal indicating the presence of the second analyte in the sample, and
if the second immobilized molecule remains unbound by the second analyte after the sample is applied to the second detection region, the detection molecule does not bind to any complex and generates a null signal indicating the absence of the second analyte in the sample.

5. The assay system of claim 1, wherein the first immobilized molecule is immobilized in the second detection region and the second immobilized molecule is immobilized in the first detection region.

6. The assay system of claim 1, wherein at least one of:
the sample is applied to the first detection region prior to the label solution being applied to the first detection region; or
the sample is applied to the second detection region prior to the label solution being applied to the second detection region.

7. The assay system of claim 1, wherein the first analyte is an antibody.

8. The assay system of claim 1, wherein the second analyte is a viral particle or an antigenic portion thereof.

9. The assay system of claim 1, wherein the first immobilized molecule comprises:
a protein, an antibody, an antigen-binding fragment of an antibody, an antigen, a peptide, a nucleic acid, or a combination thereof; or
any molecule that can bind a protein, an antibody, an antigen-binding fragment of an antibody, an antigen, a peptide, or a nucleic acid.

10. The assay system of claim 1, wherein the first immobilized molecule comprises a peptide that binds to an anti-SARS-Cov-2 S-protein neutralizing antibody and an angiotensin converting enzyme 2 (ACE 2) protein, and wherein the second immobilized molecule comprises a recombinant anti-SARS-Cov-2 antibody.

11. The assay system of claim 1, wherein the detection molecule comprises a binding moiety and a label moiety, wherein the binding moiety is a protein, an antibody, an antigen-binding fragment of an antibody, an antigen, or a peptide.

12. The assay system of claim 11, wherein the label moiety comprises a vat dye particle, wherein the vat dye particle comprises isatin, vat red 1, vat red 41, or vat orange 7.

13. The assay system of claim 12, wherein the vat dye particle is below a threshold size.

14. The assay system of claim 12, wherein:
the vat dye particle has a positively charged hydrophilic group and the binding moiety is treated to have a negative charge; or
the vat dye particle has a negatively charged hydrophilic group and the binding moiety is treated to have a positive charge.

15. The assay system of claim 11, wherein each detection molecule has more than one label moiety attached to one binding moiety.

16. A method of detecting presence or absence of at least a first analyte and a second analyte in a sample using an assay system, the method comprising:
applying the sample in a vertical direction perpendicular to a longitudinal axis of an assay device to a first detection region of the assay device, wherein the sample flows from the first detection region in a horizontal direction parallel to the longitudinal axis to a second detection region of the assay device; and
applying a label solution in the vertical direction to the first detection region, wherein the label solution comprises a detection molecule and the label solution also flows from the first detection region in the horizontal direction to the second detection region,
wherein the assay device comprises:
a first immobilized molecule immobilized in one of the first and second detection regions and configured to bind to either the detection molecule or the first analyte to indicate the presence or the absence of the first analyte in the sample; and
a second immobilized molecule immobilized in the other one of the first and second detection regions and configured to bind to the second analyte to generate a complex, wherein the detection molecule is configured to bind to the complex to indicate the presence or the absence of the second analyte in the sample.

17. The method of claim 16, wherein the first immobilized molecule is immobilized in the first detection region and the second immobilized molecule is immobilized in the second detection region.

18. The method of claim 17, wherein:
if the first immobilized molecule remains unbound by the first analyte after the sample is applied to the first detection region, the method further comprises detecting a detectable signal generated by the detection molecule binding to the first immobilized molecule indicating the absence of the first analyte in the sample, and
if the first immobilized molecule binds to the first analyte after the sample is applied to the first detection region, the method further comprises detecting a null signal generated by the detection molecule not binding to the first immobilized molecule indicating the presence of the first analyte in the sample.

19. The method of claim 17, wherein:
if the second immobilized molecule binds to the second analyte to generate the complex after the sample is applied to the second detection region, the method further comprises detecting a detectable signal generated by the detection molecule binding to the complex indicating the presence of the second analyte in the sample, and
if the second immobilized molecule remains unbound by the second analyte after the sample is applied to the second detection region, the method further comprises detecting a null signal generated by the detection molecule not binding to any complex indicating the absence of the second analyte in the sample.

20. The method of claim 16, wherein applying the sample and the label solution to the first detection region comprises applying the sample to the first detection region prior to applying the label solution to the first detection region.


SEQUENCE LISTING (3 sequences)

SEQ ID NO: 1 - protein, 805 aa, synthetic construct [sequence omitted]

SEQ ID NO: 2 - protein, 223 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 3 - protein, 419 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNLANNAAIVLQLPQGTTLPKGFYAEGSRGGSQASSRSSSRSRNSSRNSTPGSSKRTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA